(19) United States
(12) Patent Application Publication
(10) Pub. No.: US 2026/0294334 A1
(43) Pub. Date: Oct. 1, 2026

(54) ADAPTIVE SLEEP STAGE DETECTION

(71) Applicant: Edwin Ehsan Kay, Laurel, MD (US)

(72) Inventor: Edwin Ehsan Kay, Laurel, MD (US)

(21) Appl. No.: 19/633,972

(22) Filed: Mar. 30, 2026

Related U.S. Application Data
(60) Provisional application No. 63/780,455, filed on Mar. 30, 2025.

(51) Int. Cl. A61B 5/00; A61B 5/11

(52) U.S. Cl. CPC A61B 5/4812; A61B 5/11; A61B 5/4815; A61B 5/7221; A61B 5/7264; A61B 5/7282

(57) ABSTRACT

The embodiments disclose an adaptive system and method for determining sleep states using multimodal physiological data are disclosed. Physiological signals, including photoplethysmography and motion data, are processed to generate sleep stage probability distributions across dynamically determined time intervals. In response to detecting physiological transient events, the system re-segments selected intervals into shorter sub-epoch windows to increase temporal resolution and reduce signal distortion. The system modifies sleep stage probability distributions and transition behavior within the window and performs probabilistic sequence optimization to determine a most-likely sequence of sleep states. The method improves classification accuracy by integrating user-validated events with signal-driven models and supports adaptive control of sensor operation.

### BACKGROUND

Sleep plays a crucial role in human health and well-being, with sleep disorders affecting a significant portion of the population. The architecture of sleep, consisting of different stages, is vital for both physical and mental recovery. Disruptions in the temporal arrangement of these stages, seen in various sleep disorders, can negatively impact overall health. To standardize sleep staging, the American Academy of Sleep Medicine (AASM) developed Guidelines based on the R&K scoring system by Allan Rechtschaffen and Anthony Kales. These Guidelines classify sleep into well-established stages, including wake, non-REM stages (N1, N2, N3/N4), and REM sleep. However, the traditional methods of sleep staging, while effective, face challenges in terms of accuracy, cost, and time. Polysomnographic (PSG) equipment, considered the gold standard for sleep monitoring, records detailed physiological data, such as EEG and EOG. However, the manual classification of sleep stages is time-consuming, costly, and prone to variability, with experts often reaching only 80-82% agreement.

### SUMMARY OF THE INVENTION

This disclosure introduces an advanced method and system for accurately determining sleep stages by leveraging multi-sensor data, including physiological signals such as PPG (photoplethysmogram), EDA (electrodermal activity), skin temperature, heart rate, and motion, as well as non-physiological data like ambient temperature of the sleeping environment and/or contextual and user-provided data and events, including timestamped user-generated event signals indicative of sleep-related events, wherein such events are used to modify sleep stage probability distributions and transition behavior within localized temporal windows. recorded by user or by the means of other sensors or devices.

In conventional sleep staging systems, user inputs are used solely for labeling, annotation, or post hoc correction and do not alter the underlying inference model. In contrast, the disclosed system modifies the structure of the probabilistic inference process in response to user-generated event signals by generating a localized transition model constrained to a temporal window and re-optimizing the sleep state sequence within that window. This approach enables selective correction of state transitions without requiring retraining of the global model and reduces propagation of classification errors across unrelated time intervals.

Conventional systems that incorporate user annotations rely on retraining, post-processing, or labeling approaches that do not alter the probabilistic structure of the inference model. Such approaches either propagate corrections globally across an entire sleep sequence or require recomputation of model outputs over all time intervals. These methods are unable to selectively correct localized transition errors without affecting unrelated portions of the sleep sequence. In contrast, the disclosed system modifies transition behavior and sleep state likelihoods within a constrained temporal window associated with a user-generated event signal, thereby enabling localized correction while preserving global model consistency and avoiding retraining.

The system captures raw or processed sensor waveforms from various sensors, such as wearable and/or non-wearable devices. If available, the system may take advantage of medical-grade equipment. The system may continuously, occasionally, or periodically monitor sleep stages across multiple epochs during one or more sleep sessions. It extracts relevant features from each sleep epoch, such as signal amplitude, frequency, and variability, and applies feature preprocessing techniques such as weighting, feature clustering, dimensionality reduction, and feature importance determination algorithms to emphasize the most important features for accurate classification.

The system determines, for each time interval, a plurality of sleep stage probability distributions corresponding to one or more candidate sleep stages. These probability distributions represent likelihoods that the subject is in each respective sleep stage such as wake, NREM, or REM. In certain embodiments, the system adjusts these probability distributions based on temporal relationships between adjacent time intervals, including enforcing temporal consistency and reducing improbable transitions. Such adjustments may be performed using probabilistic models that account for dependencies across neighboring intervals. In a preferred embodiment, the system dynamically adjusts the temporal resolution of these intervals. Upon detecting a physiological transient event, defined by a threshold-crossing correlation or a temporal misalignment between a PPG-derived metric (e.g., peak-to-peak interval) and a motion-derived metric (e.g., accelerometer magnitude); the system re-segments a standard size (e.g., 30-second) primary interval into one or more sub-epoch windows (e.g., 2-10 seconds). This allows the model to isolate motion artifacts or rapid state transitions that would otherwise be averaged out in a standard epoch, thereby improving classification accuracy during volatile sleep periods.”

In certain embodiments, the plurality of sleep stage probability distributions may be modified in response to a user-generated event signal by increasing probabilities of states consistent with the event and decreasing probabilities of inconsistent states within a defined temporal window.

In certain embodiments, the system modifies both (i) sleep stage probability distributions and (ii) transition probabilities between sleep states in response to the user-generated event signal.

The system employs one or more computational models, including machine learning models and/or mathematical models representing sleep behavior, to process the extracted features. Each model generates an output corresponding to sleep stage probabilities. In certain embodiments, the system combines outputs from multiple models using a confidence-weighted aggregation mechanism, wherein each model output is assigned a weight based on a confidence score associated with that model. The confidence score may be determined based on signal quality, model reliability, or consistency with prior time intervals. The aggregation mechanism produces a combined probability distribution used to determine a sleep stage for each time interval.

The system employs one or more computational models, including machine learning models and/or mathematical models representing sleep behavior, to process the extracted features. Each model may generate an independent output corresponding to sleep stage probabilities. In certain embodiments, outputs from multiple models are combined using an aggregation mechanism that assigns weights to each.

The system is capable of classifying at least one sleep stage from a variety of stages over continuous time intervals or across multiple epochs within a single sleep session or across multiple sleep sessions. Each sleep session may occur at any time of day or night. By utilizing data from various sensors, including medical devices and at least one wearable device, the system continuously adapts and improves its predictions, enabling real-time analysis or retrospective analysis at the end of each sleep session, and providing personalized sleep stage classification. This innovative approach overcomes the limitations of traditional methods, offering a more efficient, accurate, and scalable solution for sleep health monitoring and assessment.

In certain embodiments, the system identifies one or more intermediate or latent sleep states that are not explicitly defined within conventional sleep staging frameworks.

These latent states may correspond to transitional physiological conditions between predefined sleep stages. The system may determine such states based on patterns in the physiological data, including clustering of feature representations or detection of states that do not satisfy criteria for predefined stages. In some embodiments, the system assigns a time interval to a latent state when the corresponding feature representation deviates from known sleep stage patterns beyond a threshold.

In certain embodiments, the system identifies latent or non-standard sleep states that are not explicitly defined by conventional sleep staging frameworks. These latent states may represent transitional or intermediate physiological conditions. The system may map physiological data into a feature space and apply clustering, probabilistic modeling, or representation learning techniques to identify latent state clusters.

A current physiological state may be classified as a latent state when its corresponding feature representation lies within a threshold distance, such as a Mahalanobis distance, from a latent cluster.

The method involves several key steps: First, continuous, occasional or periodic sleep epochs are measured from the subject using sensors that capture physiological data such as PPG, EDA, skin temperature, and others. These data points form a sequence of sleep epochs that are analyzed to gain insight into the subject's sleep patterns. Next, at least one feature, such as signal amplitude, frequency, and variability, is extracted from each time interval by analyzing the raw or processed sensor waveforms, which provides meaningful data for classification.

In certain embodiments, the system identifies physiological transient events based on interactions between multiple physiological signals. For example, a transient event may be detected based on a correlation or divergence between a PPG-derived metric, such as heart rate or pulse waveform characteristics, and a motion-derived metric obtained from accelerometer or gyroscope data.

The system may compute a cross-correlation or relative shift between these signals and determine that a transient event has occurred when the correlation exceeds or falls below a dynamically determined threshold. Such transient events may correspond to sleep stage transitions, micro-arousals, or other physiological changes. Upon detection of a transient event, the system may dynamically re-segment the corresponding time interval into one or more sub-intervals having shorter duration to enable higher-resolution analysis.

In certain embodiments, the user-generated event signal is generated in response to user input identifying a sleep-related event including at least one of attempted sleep onset, final awakening, perceived awakening during sleep, bathroom break, disturbance, nightmare, medication intake, or manual correction of a detected sleep-related event. The user input may be received via a wearable device interface, a mobile application, a voice input, or a gesture-based input detected by an inertial sensor.

The system segments the physiological data into a plurality of time intervals. In certain embodiments, the duration of one or more time intervals is dynamically determined based on characteristics of the physiological data. For example, the system may reduce the duration of a time interval in response to detecting a rapid change in physiological signals and may increase the duration during periods of relative stability. In some embodiments, dynamic segmentation is triggered by detecting changes in one or more physiological signals, including heart rate variability, motion, respiratory rate, or signal stability.

These extracted features are then fed into at least one sleep model, which may be based on machine learning algorithms trained on large datasets, mathematical models of sleep patterns, or models developed using thresholds applied to various aspects of the input signal features. Each model may use a different set of features, highlighting distinct physiological sleep characteristics. The system calculates probabilities for one or more sleep stages for each epoch, depending on the quality of the signals and the sufficiency of the data.

In certain embodiments, the system determines a signal quality metric for one or more physiological signals, including photoplethysmography signals. The signal quality metric may be computed based on one or more factors, including signal-to-noise ratio, motion artifacts, sensor contact quality, or signal stability.

The system may use the SQI to dynamically adjust the contribution of features derived from physiological signals when determining sleep stage probabilities. For example, features derived from signals with lower SQI may be down-weighted or attenuated, while features from high-quality signals may be emphasized.

This signal quality-aware weighting mechanism improves robustness of sleep stage classification in the presence of noise, motion, or sensor degradation.

The system employs a multi-model fusion architecture that reconciles a physiological model with a circadian model. The system calculates a confidence score based on the Signal Quality Index (SQI). When the SQI is high (e.g., the device is worn snugly and motion is low), the system dynamically attenuates the weight of the circadian model, prioritizing live sensor data. Conversely, when the SQI falls below a threshold (indicating noise or sensor instability), the system increases the weight of the circadian model to provide a ‘fail-safe’ prediction based on the user's historical circadian phase estimate.

These probabilities are adjusted over time using a sequence optimization process. In response to a timestamped user-generated event signal (e.g., a manual “wake”, “bed-time”, “nightmare”, tag), the system defines a localized temporal buffer window (e.g., 20 minutes) surrounding the event. Within this window, the system generates a localized transition probability model specific to the temporal window, wherein the localized transition probability model modifies transition probabilities relative to a global transition model only within the temporal window and biases transitions toward a user-indicated state. A probabilistic smoothing process, such as a Viterbi decoding algorithm, is executed over the localized temporal window to recalculate a most likely sequence of hidden sleep states. The updating of the sleep stage sequence is performed without modifying the global transition model outside the temporal window.

In certain embodiments, the system modifies sleep stage transition behavior based on user-generated or system-detected events. A temporal window surrounding such an event may be defined, and a localized transition model may be generated for that window.

The localized transition model may comprise a transition probability matrix that differs from a global transition model used for the overall sleep session. The transition probabilities within the localized model may be adjusted based on the event signal.

The localized transition probability model differs from a global transition model used for the overall sleep session by selectively modifying transition probabilities only within the temporal window associated with the user-generated event signal, while maintaining the global transition model unchanged outside the temporal window. The system may update sleep stage sequences within the temporal window using a probabilistic smoothing process, including sequence optimization algorithms such as Viterbi decoding, forward-backward inference, or other probabilistic inference methods.

The updating of the sleep stage sequence within the temporal window is performed without retraining underlying model parameters and without recomputing the sleep stage sequence outside the temporal window.

In certain embodiments, the system modifies sleep stage probability distributions based on the signal quality metric. In particular, the system attenuates contributions of features derived from signals having lower signal quality and emphasizes contributions of features derived from signals having higher signal quality. Such modification is performed in response to determining that the signal quality metric falls below or exceeds a threshold.

The user-generated event signal is not used solely as an annotation or override, but is incorporated as a constraint that modifies transition probabilities and sleep state likelihoods within the temporal window associated with the event.

This approach allows the system to continuously adapt to varying conditions and improve its predictions in real-time or after the completion of one or more sleep sessions, while also considering historical and stored data from the same or similar users to enhance accuracy. By combining multiple models, contextual insights, and user-specific data and events, the system offers a more efficient, accurate, and scalable solution for sleep health monitoring. It provides personalized sleep stage classification and may also offer the likelihood of each classified stage, both during and after sleep sessions. Additionally, the system may provide sleep quality scores and other sleep-related metrics based on statistics and insights provided by the sleep post-processing algorithms that utilize the classified stages over a period of sleep epochs or the entity of one or multiple sleep sessions.

In certain embodiments, the system computes a sleep continuity metric based on the frequency and distribution of transitions between sleep states across time intervals. The sleep continuity metric may be compared to a personalized baseline derived from historical sleep data. Deviations from the baseline may indicate disrupted or irregular sleep patterns.

The system may generate alerts, recommendations, or feedback signals, including haptic notifications or user interface outputs, when the sleep continuity metric falls outside acceptable thresholds.

In certain embodiments, the system receives user-generated or system-detected event signals indicative of sleep-related events. Upon receiving such an event signal, the system defines a temporal window surrounding the event and modifies sleep stage determination within that window by adjusting transition probabilities, enforcing or suppressing state transitions consistent with the event type, and re-optimizing the sleep stage sequence using a probabilistic sequence optimization process. The modification may include adjusting probabilities, reclassifying time intervals, or updating transition behavior in response to the event signal.

The localized modification of sleep stage determination limits propagation of classification errors beyond the temporal window and improves temporal consistency of the sleep stage sequence.

Methods, systems, and devices for automatic detection and monitoring of sleep stages are disclosed. A system may receive physiological data associated with a user from a wearable sensor device, wherein the physiological data is collected over a predetermined fixed or dynamic time interval. The system may process the physiological data by extracting at least one feature, performing signal transformations, and classifying the extracted features using a machine learning classifier to determine at least one sleep stage from a set of sleep stages.

The system may include at least one sensor configured to collect physiological signals, a processor coupled to the sensor, and a memory device storing instructions that, when executed, cause the processor to preprocess the data, extract a set of features from the physiological signals, and classify the extracted features using a machine learning model to determine sleep stages.

In certain embodiments, the system may modify operational parameters of one or more sensors based on detected sleep states or physiological conditions. Such parameters may include sampling frequency, signal resolution, or power mode.

For example, during periods of stable sleep, the system may reduce sampling frequency to conserve power, while during detected transient events or transitions, the system may increase sampling resolution or frequency.

In some implementations, the system may trigger feedback mechanisms, including haptic signals or environmental control signals, in response to detected sleep conditions.

In certain embodiments, the system modifies one or more operational parameters of the wearable device based on detected sleep states or physiological conditions. Such modification is performed in response to determining a sleep state or detecting a physiological transition. The operational parameters may include sampling frequency, signal resolution, or power mode. For example, the system may reduce sampling frequency during periods of stable sleep and increase sampling resolution during detected transitions.

The classification process may be performed locally on the wearable device or transmitted to a remote processing system for execution. The system may further generate and display an indication of the detected sleep stages, sleep stage probability scores, and associated sleep metrics via a graphical user interface of a user device. Sleep stage predictions and sleep stage transitions may be dynamically adjusted in real time or modified retrospectively, with or without additional physiological signals collected separately from the same and/or other devices. The system may incorporate adaptive learning mechanisms to refine classification accuracy over time, integrating multimodal sensor fusion and probabilistic models to enhance sleep stage transitions.

In addition to wearable sensor devices, the system may integrate sensor data from other devices to track ambient environmental information, such as temperature, humidity, light, and noise levels, to improve sleep quality assessments. Further, medical-grade devices, including wearable and non-wearable, mmWave, radar, and other contact-based or contactless sensing technologies, may be utilized to enhance the accuracy of sleep stage detection and overall sleep quality predictions. Historical physiological data collected from the user's wearable device and/or other sources may be leveraged to improve predictions. Sleep activity predictions may be adjusted based on aggregated data over short or long intervals, further enhancing the system's accuracy and adaptability.

In some embodiments, the invention relates to systems and methods implemented using one or more computing devices, processors, sensors, and communication systems configured to collect, process, analyze, and generate output data based on one or more data inputs. The system may include wearable devices, mobile devices, computers, servers, databases, sensors, cameras, microphones, and other electronic devices configured to collect physiological data, environmental data, audio data, image data, motion data, or other data. The collected data may be processed using one or more algorithms, statistical models, artificial intelligence models, or machine learning models to generate classifications, detections, predictions, comparisons, alerts, recommendations, control signals, or other outputs.

In some embodiments, the system may operate across multiple interconnected devices and may perform data collection, data processing, data storage, and data analysis locally on a device, remotely on a server, or across a distributed computing system. The system may include user interface devices configured to present outputs to a user and receive user inputs, and the system may be customized or configured based on the type of data collected, the type of sensors used, the desired outputs, and the practical application of the system.

In certain embodiments, the present disclosure relates to systems and methods implemented using one or more computing systems comprising tangible, physical hardware components including one or more processors, processing circuits, memory devices, communication interfaces, and one or more input and output devices configured to acquire, sense, measure, detect, transform, process, analyze, and generate data representative of one or more physical, environmental, biological, mechanical, or user-associated states. The computing systems may include, without limitation, wearable devices, mobile devices, smartphones, tablet computers, desktop computers, laptop computers, embedded systems, edge computing devices, Internet-of-Things (IoT) devices, smart home devices, automotive systems, industrial control systems, servers, cloud computing platforms, and combinations or distributed arrangements thereof. In certain embodiments, the processors may include one or more central processing units (CPUs), graphics processing units (GPUs), tensor processing units (TPUs), digital signal processors (DSPs), field-programmable gate arrays (FPGAs), application-specific integrated circuits (ASICs), or system-on-chip (SoC) architectures configured to execute machine-readable instructions and perform signal processing and data transformation operations.

The input devices may include one or more sensors and data acquisition components configured to generate electrical, optical, acoustic, electromagnetic, or digital signals corresponding to measurable phenomena. Such sensors may include, without limitation, physiological sensors, biometric sensors, environmental sensors, optical sensors, imaging devices such as cameras or scanners, depth sensors, infrared sensors, audio sensors such as microphones, motion sensors such as accelerometers, gyroscopes, or inertial measurement units (IMUs), proximity sensors, pressure sensors, location sensors such as global positioning system (GPS) modules, radio-frequency identification (RFID) readers, or other sensing devices configured to capture data associated with a user, an object, or an environment. The output devices may include displays, graphical user interfaces (GUIs), speakers, haptic feedback devices, actuators, control systems, or other devices configured to present information or initiate physical actions in response to generated outputs.

The communication interfaces may include wired or wireless transceivers configured to communicate data between devices over one or more communication networks, including local area networks (LANs), wide area networks (WANs), cellular networks, satellite networks, Internet-based networks, or short-range communication protocols including WiFi, Bluetooth, Bluetooth Low Energy (BLE), near-field communication (NFC), ultra-wideband (UWB), Zigbee, or other radio-frequency communication systems. In certain embodiments, communication may be facilitated using network identifiers including Internet Protocol (IP) addresses, media access control (MAC) addresses, device identifiers, session identifiers, or other addressing schemes for routing, synchronization, and coordination of data transmissions.

The computing systems may be configured to receive raw input signals from the input devices and to perform one or more physical and computational transformations on such signals. Such transformations may include analog-to-digital conversion, signal conditioning, filtering, denoising, baseline correction, normalization, scaling, time alignment, synchronization across multiple data streams, segmentation into discrete time intervals or data structures, feature extraction, encoding into vector representations, dimensionality reduction, compression, or other data transformation operations that convert raw sensor or input data into structured, machine-interpretable data representations. These structured representations may correspond to time-series data, spatial data, image data, audio data, or multi-modal data.

The structured data may then be processed using one or more computational models, including deterministic algorithms, statistical models, rule-based systems, or artificial intelligence and machine learning models, to generate outputs that correspond to a practical application. Such outputs may include classifications, detections, identifications, predictions, correlations, anomaly detections, comparisons with stored data, control signals for external systems, alerts, recommendations, or other actionable outputs that are used to control, modify, or influence a physical system, user interface, or decision-making process.

### BRIEF DESCRIPTION OF THE DRAWINGS

FIG. 1 shows, for illustrative purposes only, an example of a system that supports sleep staging algorithms in accordance with aspects of one embodiment.

FIG. 2 shows, for illustrative purposes only, an example of a wearable apparatus used to collect sensor data and signals for use with the sleep stage detection system of one embodiment.

FIG. 3 shows for illustrative purposes only an example of a user device that communicates with one or more wearable devices, as well as other possible sensor and non-sensor devices and/or medical databases, all of which support the algorithm(s) and methodology of one embodiment.

FIG. 4 shows, for illustrative purposes only, an example of graphics displaying how the outputs of a sleep analysis may be presented in a user interface of one embodiment.

FIG. 5 shows, for illustrative purposes only, an example of a sleep summary interface presenting statistical information derived from a sleep session of one embodiment.

FIG. 6 shows, for illustrative purposes only, an example of a sleep analysis application, deployable with components distributed across one or more connected systems described in this disclosure, such as a wearable device, gateway device, and/or server(s) of one embodiment.

FIG. 7 shows, for illustrative purposes only, an example of a sleep staging system pipeline of one embodiment.

FIG. 8 shows, for illustrative purposes only, an example of a multi-model sleep algorithm, outlining examples of internal model structures and an output aggregator of one embodiment.

### DETAILED DESCRIPTION

Some wearable devices, such as wrist-worn devices, are designed to collect data related to movement and other activities. These devices are capable of detecting when a user is asleep and continuously monitoring sleep patterns over a 24-hour period, encompassing both day and night. Furthermore, these wearable devices can classify different sleep stages based on the collected data, providing valuable insights into the user's sleep behavior.

Aspects of the present disclosure describe methods for automatically classifying sleep stages using data collected by wrist-worn wearable devices. The system receives physiological data, either in raw or processed formats, such as temperature, photoplethysmography (PPG) in one or more wavelength channels, electrodermal activity (EDA), heart rate, heart rate variability (HRV), SpO2, and respiratory rate, all gathered by the wearable device. This data is used to detect when the user is asleep and classify those periods into various sleep stages, including wakefulness, light sleep, REM sleep, and deep sleep. By doing so, the wearable device tracks and categorizes the user's sleep throughout its various stages.

In certain embodiments, the system operates according to a sequence of steps comprising: (i) segmenting physiological data into dynamically determined time intervals; (ii) detecting physiological transient events based on cross-modal signal relationships; (iii) re-segmenting selected intervals into sub-epoch windows for higher temporal resolution; (iv) computing signal quality metrics and adjusting feature contributions accordingly; (v) generating sleep stage probability distributions; and (vi) refining sleep stage sequences using probabilistic temporal models.

The system implementing this method may include all or a subset of key components, including: 1) sensors, which are responsible for capturing the physiological data mentioned earlier. These sensors can be integrated into the wearable device(s) or embedded in other devices, whether wearable or non-wearable, that are deployed to measure the ambient sleep environment. 2) a processing unit, which analyzes the sleep epochs using one or more machine learning classifiers, potentially combined with other mathematical modeling systems and analytics. This allows the system to adaptively classify sleep stages based on the extracted features, generate sleep classifications, assign sleep scores, and determine relevant health metrics with their associated probabilities. 3) a user interface, which presents the classified sleep stages and provides insights into the user's health metrics, either in real-time or retrospectively by analyzing one or multiple sleep epochs or sessions. The interface can summarize the data in graphical formats, tables, or other visual representations, offering feedback on sleep quality, and may provide personalized summaries and recommendations for improving sleep patterns.

In the context of the disclosed system, a “time interval” refers to a specific, predefined period during which physiological data is captured and analyzed. The length of this time interval may be fixed or dynamic, depending on the specific requirements of the sleep model and the nature of the physiological signals being studied. For example, in certain implementations, the time interval may be a fixed duration, such as 30 seconds or 1 minute, during which sensor data, including heart rate variability, PPG, EDA, skin temperature, and other physiological parameters, is recorded and processed. In other instances, the time interval may be dynamic, adjusting in real-time based on detected changes in the subject's sleep behavior, environmental factors, or model requirements. Similarly, a “sleep epoch” refers to a period of time during which the subject's sleep is monitored, and it can represent either a fixed or variable duration. A sleep epoch may correspond to a specific segment of sleep during which particular sleep stages, such as light sleep, REM, or deep sleep, are evaluated. The length of each sleep epoch is typically based on the nature of the data collected, the modeling techniques used, and the desired resolution of the sleep stage classification. For example, a sleep epoch could be defined as a 30-second window in which sleep stage transitions are analyzed, or it may dynamically adapt based on real-time sleep behavior, such as changes in heart rate or movement patterns.

In the context of this disclosure, a sleep epoch refers to a continuous time segment or interval of sleep within a sleeping session. It represents a short, often predefined time window (e.g., a few seconds or minutes) during which collected data is continuously monitored and classified into specific sleep stages, such as wake, light sleep, REM sleep, or deep sleep. Multiple sleep epochs together may form a complete sleep cycle. A sleep session, on the other hand, is defined as the entire duration from the onset of sleep to its termination, which may be detected automatically, or triggered by the user activating an event button on the wearable device or through the user interface on their connected device (e.g., smartphone, tablet, or laptop). The sleep session may encompass one or more than one sleep stages experienced by the user throughout the session. The event button or app interface allows the user to mark the start and end of their sleep period, providing clear boundaries for the sleep session. Within each sleep session, at least one sleep epoch is monitored and classified using the collected data.

In some implementations, the system may adjust the length and definition of time intervals and sleep epochs dynamically, allowing for more accurate and responsive analysis based on the subject's specific sleep patterns or changes in real-time data. For instance, if the system detects a sudden change in the subject's sleep behavior, such as the onset of sleep apnea or an abnormal heart rate fluctuation, the time interval or sleep epoch may be modified to better capture the event for subsequent analysis. This dynamic adjustment provides flexibility in analyzing diverse sleep patterns, offering more precise and individualized sleep assessments.

In certain embodiments, the duration of the primary time intervals and sub-epoch windows is dynamically optimized by an artificial intelligence model (e.g., a neural network or a random forest classifier) configured to process a historical sleep architecture profile associated with the subject. The historical sleep architecture profile may comprise a statistical distribution of sleep-wake transitions, average stage durations, and circadian-linked transition densities derived from one or more prior sleep sessions. For example, during the first portion of a sleep session where the historical profile indicates a high probability of stable N3 (Deep) sleep, the AI model may increase the primary time interval (e.g., to 60 seconds) to reduce computational overhead and sensor power consumption. Conversely, during periods where the historical profile or real-time feature trends indicate a high density of transitions; such as the transition from NREM to REM sleep; the AI model may pre-emptively reduce the interval duration to a shorter sub-epoch (e.g., 5 to 15 seconds) to ensure high-fidelity capture of the state shift.

The artificial intelligence model may utilize historical patterns of sleep stage transitions, including timing, frequency, and duration of prior sleep stages, to predict an optimal temporal resolution for subsequent analysis. For example, the system may reduce the duration of time intervals during periods associated with rapid physiological transitions and increase the duration during periods associated with physiological steady-state conditions. The artificial intelligence model may further utilize the historical sleep profile to calibrate the dynamically determined threshold used for detecting physiological transient events. By analyzing past correlations between signals, such as motion and photoplethysmography (PPG) metrics for a specific subject, the system can distinguish between normal shifting during sleep and significant state transitions. This personalized thresholding reduces false-positive re-segmentation events, thereby optimizing the allocation of processing resources and improving the reliability of the assigned sleep states.

In some embodiments, the duration of time intervals may be iteratively adjusted based on real-time signal characteristics, including entropy, variance, or rate-of-change of physiological signals, thereby enabling improved temporal precision and computational efficiency.

In some embodiments, the system presents the classified sleep stages to the user through a graphical user interface (GUI) on a user device, such as a smartphone or tablet. The GUI may display time intervals during sleep, with segments labeled or otherwise marked with the corresponding sleep stages. This classification process provides valuable feedback to users, such as recommendations for bedtimes, wake-up times, or insights into overall sleep patterns.

To improve the accuracy of sleep stage classification, the system may incorporate a machine learning classifier that analyzes the physiological data collected by at least one wearable device. The machine learning model is trained to detect specific patterns. Some wearable devices, such as wrist-worn devices, are designed to collect data related to movement and other activities. These devices can detect when a user is asleep and monitor sleep patterns continuously over a 24-hour period, including both day and night. Additionally, these wearable devices can classify different sleep stages based on the collected data, providing valuable insights into the user's sleep behavior.

The present disclosure describes methods for automatically classifying sleep stages using data collected by wrist-worn wearable devices. The system receives physiological data, in either raw or processed formats, such as temperature, photoplethysmography (PPG) in one or multiple wavelength channels, electrodermal activity (EDA), heart rate, heart rate variability (HRV), SpO2, and respiratory rate, gathered by the wearable device. This data is used to detect when the user is asleep and to classify those periods into various sleep stages, such as wakefulness, light sleep, REM sleep, and deep sleep. This allows the wearable device to track and categorize the user's sleep throughout its different stages.

To improve the accuracy of sleep stage classification, the system may incorporate a machine learning classifier that analyzes the physiological data collected by the wearable device. The machine learning model is trained to detect specific patterns and features in the data, such as the rate of change in a parameter or the relationships between physiological measures. The classifier can be personalized for each user by training it with the user's unique sleep data, optimizing the system to better match individual sleep patterns.

In addition, certain implementations can leverage circadian rhythm-derived features to further enhance sleep stage classification. Circadian rhythms regulate an individual's natural sleep-wake cycle, typically repeating every 24 hours. The system may include a circadian rhythm adjustment model that fine-tunes the physiological data, improving classification accuracy. This model can be tailored to each user based on their personal sleep patterns, providing a more precise reflection of the user's sleep cycle.

In certain embodiments, the system incorporates a circadian model that generates a circadian phase estimate based on historical and contextual data. The circadian model may operate in conjunction with a physiological model that processes real-time physiological signals.

The system may combine outputs from the physiological model and the circadian model using a confidence-weighted aggregation mechanism. A confidence score for the physiological model may be determined based on real-time signal quality metrics. The contribution of the circadian model may be dynamically attenuated when the confidence score of the physiological model exceeds a threshold, thereby prioritizing real-time physiological measurements over predicted circadian patterns.

In certain embodiments, the system processes contextual data using a circadian model to generate a circadian phase estimate. The system combines outputs from the circadian model and a physiological model using a confidence-weighted aggregation mechanism. A confidence score for the physiological model is determined based on real-time signal quality. The system dynamically attenuates the contribution of the circadian model when the confidence score of the physiological model exceeds a threshold, thereby prioritizing real-time physiological measurements over predicted circadian patterns.

In various configurations, the system operates as part of an interconnected network, where wearable devices, user devices (e.g., smartphones, laptops), and servers collaborate to collect, process, and analyze all provided physiological, ambient, and/or user medical and non-medical information to classify sleep stages. This interconnected setup allows the system to provide users with accurate, actionable insights into their sleep stages and overall sleep health.

In some implementations, a user-programmable event button may be integrated into the wearable device, enabling users to mark specific sleep-related events. For example, users can press the button to indicate the start or end of a sleep episode, occasional wake times during the night, or other sleep-related occurrences. The event button may support multiple functions, such as a single press to mark sleep onset or wake-up time, a double press to indicate an interrupted sleep event, and a long press to capture user input regarding sleep quality or disturbances, such as discomfort or environmental noise.

User-inputted event data can enhance sleep stage classification by providing context to physiological data. For instance, if a user marks a wake event during the night, the system can refine its sleep stage detection models to improve accuracy. Event button inputs may also allow the system to incorporate additional context, such as the user's mood, fatigue level compared to previous sleep sessions, or their life status (e.g., recent overexertion, sleep deprivation, travel, or jet lag).

In one embodiment, the signals received from the event button may serve as an input to the sleep staging algorithm, contributing to the classification and refinement of sleep stage transitions. The event button may be manually activated by the user to indicate specific sleep-related occurrences, such as awakenings, perceived sleep paralysis episodes, or disturbances during sleep.

The user-generated event signal is not incorporated as an input feature to the probabilistic model and is not used for labeling or training. Instead, the event signal is applied as a constraint that modifies transition probabilities and state likelihoods within a defined temporal window of the inference process.

In certain embodiments, the system may generate a prompt in response to detecting a candidate event from physiological signals, including motion bursts or signal disruptions, and may receive a user confirmation or correction. The confirmed or corrected input is used to generate the user-generated event signal.

In certain embodiments, the user-generated event signal is not incorporated as an input feature for model training or classification, but is applied as a constraint that modifies transition probabilities and state likelihoods within a temporal window of the probabilistic inference process.

In another embodiment, the event button data may be used for feedback mechanisms, where the system dynamically adjusts algorithm parameters based on user-reported events. For example, if a user presses the event button during a perceived sleep paralysis episode, the system may prioritize analyzing muscle atonia, heart rate variability, and respiratory patterns during that period to improve detection and personalized insights.

In another embodiment, the event button signals may be integrated into a longitudinal analysis framework, where the system identifies recurring sleep disturbances over multiple nights. This data may assist in clinical evaluations, allowing healthcare providers to correlate self-reported experiences with physiological evidence, thus enabling tailored treatment recommendations.

In some configurations, the system may integrate information from sensor-based or non-sensor-based devices that provide insights into the ambient sleep environment or other medical and non-medical databases. These databases may offer additional valuable data about the user's daily activity, medical records (with user or practitioner consent), or insights from a group of users with similar characteristics.

In some implementations, all wearable and non-wearable sensors, as well as databases, may be connected to the user's device(s) 105 (e.g., laptop or tablet) to carry out the sleep staging analysis. Alternatively, these devices and sensors may be connected to one or multiple servers through an internal network or internet connection, enabling data storage and performing all the sleep analyses described in this disclosure.

Some wearable devices, such as wrist-worn devices, are designed to collect data related to movement and other activities. These devices are capable of detecting when a user is asleep and continuously monitoring sleep patterns over a 24-hour period, encompassing both day and night. Furthermore, these wearable devices can classify different sleep stages based on the collected data.

Aspects of the present disclosure describe methods for automatically classifying sleep stages using data collected by wrist-worn wearable devices. The system receives physiological data, either in raw or processed formats, such as temperature, photoplethysmography (PPG) in one or more wavelength channels, electrodermal activity (EDA), heart rate, heart rate variability (HRV), SpO2, and respiratory rate, all gathered by the wearable device.

This data is used to detect when the user is asleep and classify those periods into various sleep stages, including wakefulness, light sleep, REM sleep, and deep sleep. By doing so, the wearable device tracks and categorizes the user's sleep throughout its various stages. The system implementing this method may include all or a subset of key components, including: 1) sensors, which are responsible for capturing the physiological data mentioned earlier.

These sensors can be integrated into the wearable device(s) or embedded in other devices, whether wearable or non-wearable, that are deployed to measure the ambient sleep environment. 2) a processing unit, which analyzes the sleep epochs using one or more machine learning classifiers, potentially combined with other mathematical modeling systems and analytics. This allows the system to adaptively classify sleep stages based on the extracted features, generate sleep classifications, assign sleep scores, and determine relevant health metrics with their associated probabilities. 3) a user interface, which presents the classified sleep stages and provides insights into the user's health metrics, either in real-time or retrospectively by analyzing one or multiple sleep epochs or sessions. The interface can summarize the data in graphical formats, tables, or other visual representations, offering feedback on sleep quality, and may provide personalized summaries and recommendations for improving sleep patterns. In the context of the disclosed system, a “time interval” refers to a specific, predefined period during which physiological data is captured and analyzed. The length of this time interval may be fixed or dynamic, depending on the specific requirements of the sleep model and the nature of the physiological signals being studied. For example, in certain implementations, the time interval may be a fixed duration, such as 30 seconds or 1 minute, during which sensor data, including heart rate variability, PPG, EDA, skin temperature, and other physiological parameters, is recorded and processed. In other instances, the time interval may be dynamic, adjusting in real-time based on detected changes in the subject's sleep behavior, environmental factors, or model requirements.

Similarly, a “sleep epoch” refers to a period of time during which the subject's sleep is monitored, and it can represent either a fixed or variable duration. A sleep epoch may correspond to a specific segment of sleep during which particular sleep stages, such as light sleep, REM, or deep sleep, are evaluated. The length of each sleep epoch is typically based on the nature of the data collected, the modeling techniques used, and the desired resolution of the sleep stage classification. For example, a sleep epoch could be defined as a 30-second window in which sleep stage transitions are analyzed, or it may dynamically adapt based on real-time sleep behavior, such as changes in heart rate or movement patterns. In the context of this disclosure, a sleep epoch refers to a continuous time segment or interval of sleep within a sleeping session. It represents a short, often predefined time window (e.g., a few seconds or minutes) during which collected data is continuously monitored and classified into specific sleep stages, such as wake, light sleep, REM sleep, or deep sleep. Multiple sleep epochs together may form a complete sleep cycle.

A sleep session, on the other hand, is defined as the entire duration from the onset of sleep to its termination, which may be detected automatically, or triggered by the user activating an event button on the wearable device or through the user interface on their connected device (e.g., smartphone, tablet, or laptop). The sleep session may encompass one or more than one sleep stages experienced by the user throughout the session.

The event button or app interface allows the user to mark the start and end of their sleep period, providing clear boundaries for the sleep session. Within each sleep session, at least one sleep epoch is monitored and classified using the collected data. In some implementations, the system may adjust the length and definition of time intervals and sleep epochs dynamically, allowing for more accurate and responsive analysis based on the subject's specific sleep patterns or changes in real-time data. For instance, if the system detects a sudden change in the subject's sleep behavior, such as the onset of sleep apnea or an abnormal heart rate fluctuation, the time interval or sleep epoch may be modified to better capture the event for subsequent analysis.

This dynamic adjustment provides flexibility in analyzing diverse sleep patterns, offering more precise and individualized sleep assessments. In some embodiments, the system presents the classified sleep stages to the user through a graphical user interface (GUI) on a user device, such as a smartphone or tablet. The GUI may display time intervals during sleep, with segments labeled or otherwise marked with the corresponding sleep stages.

This classification process provides valuable feedback to users, such as recommendations for bedtimes, wake-up times, or insights into overall sleep patterns. This dynamic adjustment provides flexibility in analyzing diverse sleep patterns, offering more precise and individualized sleep assessments. In some embodiments, the system presents the classified sleep stages to the user through a graphical user interface (GUI) on a user device, such as a smartphone or tablet.

The GUI may display time intervals during sleep, with segments labeled or otherwise marked with the corresponding sleep stages. This classification process provides valuable feedback to users, such as recommendations for bedtimes, wake-up times, or insights into overall sleep patterns.

To improve the accuracy of sleep stage classification, the system may incorporate a machine learning classifier that analyzes the physiological data collected by at least one wearable device. The machine learning model is trained to detect specific patterns. Some wearable devices, such as wrist-worn devices, are designed to collect data related to movement and other activities. These devices can detect when a user is asleep and monitor sleep patterns continuously over a 24-hour period, including both day and night.

Additionally, these wearable devices can classify different sleep stages based on the collected data, providing valuable insights into the user's sleep behavior. The present disclosure describes methods for automatically classifying sleep stages using data collected by wrist-worn wearable devices. The system receives physiological data, in either raw or processed formats, such as temperature, photoplethysmography (PPG) in one or multiple wavelength channels, electrodermal activity (EDA), heart rate, heart rate variability (HRV), SpO2, and respiratory rate, gathered by the wearable device.

This data is used to detect when the user is asleep and to classify those periods into various sleep stages, such as wakefulness, light sleep, REM sleep, and deep sleep. This allows the wearable device to track and categorize the user's sleep throughout its different stages. In some embodiments, the system presents the classified sleep stages to the user through a graphical user interface (GUI) on a user device, such as a smartphone or tablet. The GUI may display time intervals during sleep, with segments labeled or otherwise marked with the corresponding sleep stages. This classification process provides valuable feedback to users, such as recommendations for bedtimes, wake-up times, or insights into overall sleep patterns. To improve the accuracy of sleep stage classification, the system may incorporate a machine learning classifier that analyzes the physiological data collected by the wearable device.

The machine learning model is trained to detect specific patterns and features in the data, such as the rate of change in a parameter or the relationships between physiological measures. The classifier can be personalized for each user by training it with the user's unique sleep data, optimizing the system to better match individual sleep patterns. In addition, certain implementations can leverage circadian rhythm-derived features to further enhance sleep stage classification.

Circadian rhythms regulate an individual's natural sleep-wake cycle, typically repeating every 24 hours. The system may include a circadian rhythm adjustment model that fine-tunes the physiological data, improving classification accuracy. This model can be tailored to each user based on their personal sleep patterns, providing a more precise reflection of the user's sleep cycle.

In various configurations, the system operates as part of an interconnected network, where wearable devices, user devices (e.g., smartphones, laptops), and servers collaborate to collect, process, and analyze all provided physiological, ambient, and/or user medical and non-medical information to classify sleep stages. This interconnected setup allows the system to provide users with accurate, actionable insights into their sleep stages and overall sleep health. In some implementations, a user-programmable event button may be integrated into the wearable device, enabling users to mark specific sleep-related events.

For example, users can press the button to indicate the start or end of a sleep episode, occasional wake times during the night, or other sleep-related occurrences. The event button may support multiple functions, such as a single press to mark sleep onset or wake-up time, a double press to indicate an interrupted sleep event, and a long press to capture user input regarding sleep quality or disturbances, such as discomfort or environmental noise. User-inputted event data can enhance sleep stage classification by providing context to physiological data. For instance, if a user marks a wake event during the night, the system can refine its sleep stage detection models to improve accuracy.

Event button inputs may also allow the system to incorporate additional context, such as the user's mood, fatigue level compared to previous sleep sessions, or their life status (e.g., recent overexertion, sleep deprivation, travel, or jet lag). In one embodiment, the signals received from the event button may serve as an input to the sleep staging algorithm, contributing to the classification and refinement of sleep stage transitions. The event button may be manually activated by the user to indicate specific sleep-related occurrences, such as awakenings, perceived sleep paralysis episodes, or disturbances during sleep.

These signals may be processed alongside physiological data from sensors, such as PPG, EEG, EDA, accelerometer, and skin temperature sensors. The event button input can be incorporated into machine learning models or heuristic algorithms to enhance the accuracy of sleep staging. Additionally, these signals may be leveraged by any of the algorithms implemented in the application, including sleep pattern analysis, sleep anomaly detection, or circadian rhythm optimization models.

In another embodiment, the event button data may be used for feedback mechanisms, where the system dynamically adjusts algorithm parameters based on user-reported events. For example, if a user presses the event button during a perceived sleep paralysis episode, the system may prioritize analyzing muscle atonia, heart rate variability, and respiratory patterns during that period to improve detection and personalized insights. In another embodiment, the event button signals may be integrated into a longitudinal analysis framework, where the system identifies recurring sleep disturbances over multiple nights.

This data may assist in clinical evaluations, allowing healthcare providers to correlate self-reported experiences with physiological evidence, thus enabling tailored treatment recommendations. In some configurations, the system may integrate information from sensor-based or non-sensor-based devices that provide insights into the ambient sleep environment or other medical and non-medical databases. These databases may offer additional valuable data about the user's daily activity, medical records (with user or practitioner consent), or insights from a group of users with similar characteristics. In some implementations, all wearable and non-wearable sensors, as well as databases, may be connected to the user's device(s) 105 (e.g., laptop or tablet) to carry out the sleep staging analysis. Alternatively, these devices and sensors may be connected to one or multiple servers through an internal network or internet connection, enabling data storage and performing all the sleep analyses described in this disclosure.

In certain embodiments, the system is configured to store and manage historical data associated with one or more users. Such data may include raw sensor data, processed features, sleep stage classifications, confidence scores, and derived sleep metrics. The data may be stored locally on user devices, remotely on server systems, or in distributed storage environments.

In certain embodiments, historical data may be used to personalize sleep stage classification models, including adjusting model parameters based on user-specific patterns. The system may also utilize aggregated data from multiple users to train or refine machine learning models, enabling improved accuracy across diverse populations. In certain embodiments, the system may retrieve historical or population-level data during operation to inform real-time classification decisions.

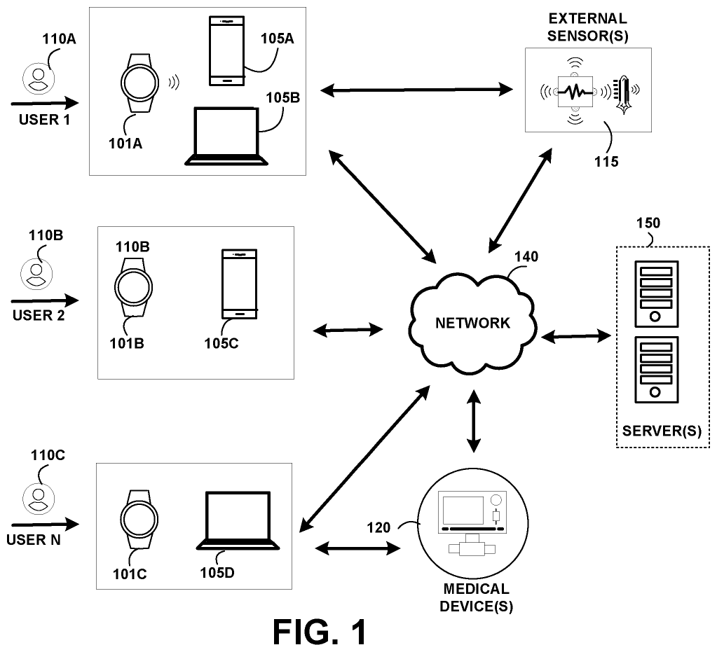

FIG. 1 shows, for illustrative purposes only, an example of a system that supports sleep staging algorithms in accordance with aspects of one embodiment. FIG. 1 shows for illustrative purposes only an example of a system 100 that supports data collection, communication, and processing, wherein the system 100 includes network user 1, user 2, and user n associated with wearable devices 101A, 101B, and 101C, respectively, and the wearable devices 101A, 101B, and 101C are configured to collect data from users 110A, 110B, and 110C and communicate with user devices 105A, 105B, 105C, and 105D. The wearable devices 101A, 101B, and 101C, the user devices 105A, 105B, 105C, and 105D, external sensor(s) 115, and medical device(s) 120 are configured to communicate through network 140, and data collected from the wearable devices 101A, 101B, and 101C, the external sensor(s) 115, and the medical device(s) 120 may be transmitted through network 140 to server(s) 150 for storage, processing, and analysis, and the server(s) 150 may transmit processed data and analysis results through network 140 to the user devices 105A, 105B, 105C, and 105D associated with network user 1, user 2, and user N. FIG. 1 illustrates an example of a system 100 that supports sleep staging algorithms in accordance with aspects of the present disclosure.

The system 100 includes a variety of electronic devices (e.g., wearable devices 101, user devices 105) that may be worn and/or operated by one or more users 101. The system 100 also includes a network 140 and one or more servers 150. The electronic devices may encompass any devices known in the art, including wearable devices 101 (e.g., wrist-worn devices such as smartwatches, fitness bands, etc.) and user devices 105 (e.g., smartphones, laptops, tablets). These devices associated with users 110 can perform multiple functions, such as: 1) measuring physiological data, 2) measuring non-physiological sensor-based or non-sensor-based data, 3) connecting to medical databases, 4) storing the collected data, 5) processing the data, 6) providing outputs (e.g., via graphical user interfaces (GUIs)) to the user 110 based on the processed data, and 7) facilitating communication of data between devices and other computing systems. Different electronic devices may execute one or more of these functions.

Wearable devices 101 may include computing devices worn by the user, such as wrist-worn devices (e.g., smartwatches, fitness bands, or bracelets) and/or head-mounted devices (e.g., glasses or goggles). Wearable devices 101 may also be configured as bands, straps (flexible or inflexible), stick-on sensors, or other wearables that can be placed in various body locations, including bands worn around the head (e.g., forehead headbands), arm (e.g., forearm or bicep bands), or leg (e.g., thigh or calf bands), or placed behind the ear, under the armpit, and so on. Additionally, wearable devices 101 could be incorporated into articles of clothing, such as jackets, shirts, gloves, socks, or undergarments. In some configurations, wearable devices 101 may be integrated into sporting or training equipment, such as bicycles, skis, tennis rackets, golf clubs, or training weights.

Although the present disclosure is described in the context of a wrist-worn wearable device 101, this is not meant to be limiting. The terms “wearable device”, “wrist-wearable device 101”, “wearable device 101,” and similar terms are used interchangeably in this context. However, it is recognized that aspects of the present disclosure may apply to a range of wearable devices, including but not limited to wrist-worn devices, necklaces, bracelets, anklets, earrings, and more.

The wearable device 101 is responsible for collecting physiological and event signals from the user. It continuously monitors at least one physiological parameter, such as photoplethysmography (PPG) for heart rate and blood oxygen levels, electrodermal activity (EDA) for stress monitoring, skin temperature for circadian rhythm tracking, heart rate variability (HRV) for autonomic nervous system analysis, and respiratory rate to assess breathing patterns over a period of time. The wearable device transmits collected data to the user's personal device 105 through wireless communication protocols such as Bluetooth, infrared, or Wi-Fi. In some implementations, a single personal device may manage multiple wearable devices simultaneously, supporting a multi-sensor ecosystem.

The personal device(s) 105 may act as a hub that facilitates communication between the wearable device and other components of the system. It can be a smartphone, tablet, laptop, or a dedicated IoT hub. This device aggregates data from multiple sources, such as wearables, medical-grade devices 120, and environmental sensors 115. It can preprocess and store data before transmitting it to a remote or local server 150 for further analysis. A user's personal device(s) 105 may also be capable of executing the entire sleep stage algorithm, depending on the complexity of the algorithm architecture and its processing power. In some implementations, multiple personal devices 105 can be orchestrated in a system. For example, one device may serve as a hub, connecting all sensor devices, collecting required data, executing the sleep classification algorithm, and sending feedback to IoT devices, while another device, such as a personal cellphone, tablet, or laptop, is used to visualize the sleep stage outputs, sleep scores, and relevant health metrics for the user.

In addition to data collection, the personal device 105 can also control other connected systems. For example, it may adjust room temperature, lighting, or sound levels based on the user's sleep patterns. A smart thermostat may automatically lower the temperature when the user enters deep sleep, or a white noise machine may be activated if background noise levels exceed a certain threshold.

Medical-grade devices 120 may provide more accurate and regulated physiological measurements that are often used in clinical or home monitoring settings. These devices may include electrocardiograms (EKG) for heart rhythm analysis, polysomnography (PSG) systems for sleep studies, continuous blood pressure monitors, and continuous glucose monitoring devices. They may either be connected to the user's personal device 105 or communicate directly with a remote or cloud server.

In some implementations, medical-grade devices may be used in conjunction with wearable devices to improve the accuracy of sleep stage classification. For example, a wearable device may track general sleep patterns, while an EKG monitor provides detailed heart activity data to refine sleep analysis. Alternatively, medical-grade devices may function independently, collecting precise physiological data without requiring a wearable device.

External edge devices 115 complement the wearable device by gathering additional environmental data. These devices measure factors such as room temperature, humidity, air quality, noise levels, and light intensity. For example, any changes in the sleeping pattern or irregularities in sleep patterns may be attributed to a dramatic change in the sleeping ambient temperature, prompting the system to activate ventilation to improve sleep quality while closely tracking sleep behavior in real-time.

In some implementations, external devices may include radar-based movement tracking sensors, millimeter-wave (mmWave) sensors, or infrared cameras to monitor user sleep behavior without requiring the user to wear a device. For example, an mmWave sensor can detect the user's movement, respiratory rate, and heart rate while sleeping. However, the accuracy and consistency of the measurements might not be as precise as those provided by medical-grade devices or wearable contact-based devices. Nevertheless, this category of devices provides valuable sleep insights, even though the accuracy may be lower than direct physiological measurements from a wearable device. In some implementations, external sensors may be embedded into the user's environment, such as being mounted on walls or ceilings. A ceiling-mounted radar sensor could track movement patterns throughout the night, detecting restlessness or potential sleep disturbances. This allows for passive sleep tracking without the need for the user to wear any device.

While non-contact systems may provide lower accuracy (e.g., 60-75%) compared to wearable devices (which can achieve 80-90%), they can still effectively improve the sleeping environment. For example, if a radar sensor detects excessive tossing and turning, the system might adjust the room temperature or recommend changes to the user's sleep routine. The insights from such non-contact systems may complement the information collected from wearables, such as data provided by motion sensors. The network 140 connects all devices, ensuring seamless communication between wearable devices, personal devices, external sensors, and medical-grade equipment. This network can operate locally within a home environment or connect to remote servers via the Internet. It aggregates data from multiple sources and facilitates secure data storage and processing. Depending on the complexity of the analysis, sleep staging algorithms and health assessments may be executed on different devices. If immediate adjustments to the environment are needed, such as controlling room temperature, simple models may run locally on the personal device 105. More complex analyses, such as detailed trend tracking and personalized sleep recommendations, may require execution on cloud-based servers.

In some implementations, the user's personal device 105 can act as a controller for other connected systems. For example, based on sleep detection, it may adjust an air conditioner, turn off the lights, or activate a wake-up alarm at an optimal time. These functions can be automated and personalized based on the user's historical sleep patterns and preferences. The execution of sleep staging algorithms may be distributed among different devices depending on processing needs. In some implementations, preprocessing tasks may be performed on the wearable device 101 before transmitting data to the personal device 105. The personal device may then generate preliminary sleep classifications, while post-processing and deeper analysis are completed on remote servers when large datasets require more intensive computation.

In some implementations, all sleep staging computations may be executed directly on the wearable device, with only the final sleep stages being transmitted to the user's personal device. This reduces the amount of transmitted data and allows for more real-time sleep tracking without the need for constant external processing. In some implementations, the wearable device may only transmit raw sensor and event data to the personal device, which then forwards it to a remote server for complete sleep analysis. In this case, the user's device primarily serves as a relay, and the final sleep report is retrieved from the server when needed. In some implementations, the wearable device 101 may be removed entirely from the system, relying solely on non-wearable sensors.

For example, a bedroom equipped with mmWave sensors, radar sensors, and infrared cameras may provide sufficient data to track sleep stages and detect sleep disturbances without requiring the user to wear a device. In some implementations, only medical-grade sensors 120 may be used as the primary source of physiological data, either supplementing wearable devices 101 or operating independently. For example, an EKG and a continuous glucose monitor may provide highly detailed physiological data for clinical sleep analysis without the need for additional sensors.

In other implementations, both wearable devices 101 and medical-grade devices 120 may be used, either with or without additional external sensors. For example, a user may wear a wristband sleep tracker while also using an external mmWave sensor to validate sleep staging data. In some cases, medical databases may also be integrated to enhance sleep analysis by comparing user data to established medical trends.

The system can adapt to different configurations based on user needs and available hardware. Whether using a combination of wearable, medical, and environmental sensors or relying on a single category of devices, the system provides a flexible and scalable approach to monitoring sleep stages.

Techniques described herein may provide for improved sleep stage classification using data collected by a wearable device. In particular, a wrist-worn device, such as Watch 101, may implement sleep staging algorithms to determine periods of time during which a respective user is engaged in specific sleep stages, including awake, light sleep, REM sleep, and deep sleep. By leveraging physiological data collected from the user, Watch 101 may provide a more comprehensive evaluation of the user's sleep patterns, enabling more accurate feedback regarding sleep quality. This detailed analysis may allow the user to make informed adjustments to their sleep habits, which may, in turn, improve overall sleep quality and general well-being.

In certain embodiments, the system comprises a distributed networked architecture including one or more user devices, external sensor devices, medical devices, and one or more remote server systems communicatively coupled via a network. The network may comprise one or more wired and/or wireless communication networks, including but not limited to the Internet, local area networks (LAN), wide area networks (WAN), cellular networks, Bluetooth communication links, or other short-range or long-range communication protocols. Each user device may be associated with a respective user and may include wearable devices, mobile devices, or computing devices configured to collect, process, and transmit physiological and non-physiological data.

In certain embodiments, external sensors may be configured to collect supplemental physiological or environmental data, including but not limited to ambient temperature, humidity, light exposure, sound levels, or other contextual parameters. These external sensors may communicate directly with user devices or indirectly via the network. In certain embodiments, medical devices, such as clinical monitoring systems, may also be integrated into the network and may provide additional physiological measurements for calibration, validation, or augmentation of sleep stage determination.

In certain embodiments, one or more server systems are configured to receive data from user devices and external sensors, perform additional processing, store historical data, and provide analytics or feedback to users or clinicians. The server systems may include cloud-based infrastructure, distributed computing environments, or centralized databases configured to support large-scale data aggregation and model training. In certain embodiments, communication between system components may be bi-directional, enabling real-time or near real-time data exchange and adaptive system behavior.

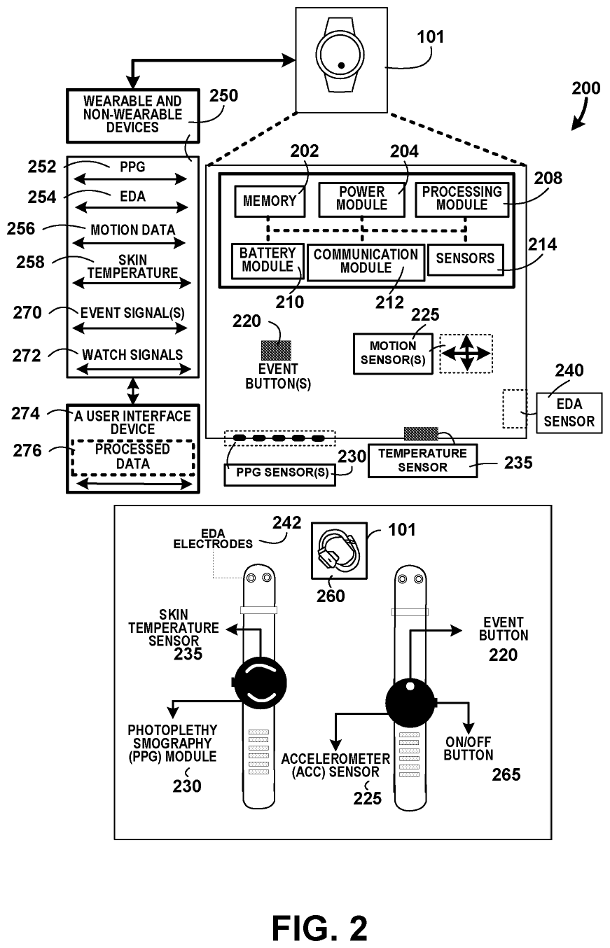

FIG. 2 shows, for illustrative purposes only, an example of a wearable apparatus used to collect sensor data and signals for use with the sleep stage detection system of one embodiment. FIG. 2 shows for illustrative purposes only an example of a wearable device 101 configured as a watch system 200, wherein the watch system 200 includes a memory 202, a power module 204, a processing module 208, a battery module 210, a communication module 212, and sensors 214, and the wearable and non-wearable devices 250 include PPG 252, EDA 254, motion data 256, skin temperature 258, event signal(s) 270, and watch signals 272, and a user interface device 274 is configured to display processed data 276, and the sensors include event button(s) 220, motion sensor(s) 225, PPG sensor(s) 230, temperature sensor 235, EDA sensor 240, and EDA electrodes 242, and the wearable device 101 further includes an ON/OFF button 265 and an accelerometer (ACC) sensor 225, wherein the memory 202, power module 204, processing module 208, battery module 210, communication module 212, and sensors 214 are configured to collect, process, store, and transmit physiological data, motion data, skin temperature data, electrodermal activity data, and event signal(s).

FIG. 2 illustrates an example of a wearable device 200 that supports sleep staging algorithms in accordance with aspects of the present disclosure. The system 200 may implement, or be implemented by, system 100. In particular, system 200 illustrates an example of a wrist-worn watch-like device, referred to herein as Watch 101, as described with reference to FIG. 1. In some aspects, Watch 101 may be configured to be worn around a user's wrist and may determine one or more physiological parameters when worn. Such parameters may include, but are not limited to, user skin temperature, PPG signals, respiratory rate, heart rate, heart rate variability, and blood oxygen levels. By continuously monitoring these physiological parameters, Watch 101 may provide real-time sleep stage classification without requiring external monitoring equipment.

It should be appreciated by a person skilled in the art that one or more aspects of the disclosure may be implemented in a system 100 to additionally or alternatively solve other problems than those described above. Furthermore, aspects of the disclosure may provide technical improvements over conventional systems or processes, including enhancements in sensor accuracy, data processing efficiency, and sleep staging precision. However, the description and appended drawings provide only example technical improvements resulting from implementing aspects of the disclosure and accordingly do not represent all of the technical advancements provided within the scope of the claims. The wearable watch may include a housing 260 comprising an upper housing and a bottom housing. In some aspects, the housing of Watch 101 may enclose various components, including but not limited to device electronics, a power source such as battery 210 or a capacitor, and one or more substrates, such as printable circuit boards that interconnect the device electronics and power source. The device electronics may include various modules, such as a processing module 208, a memory module 202, a communication module 212, and a power module 204. Additionally, the device electronics may integrate one or more sensors, including but not limited to temperature sensors 235, a PPG sensor assembly (e.g., PPG system 230), motion sensors 225, and EDA sensors 240. The watch may also include one or more event buttons 220.

The sensors may be configured to communicate with the respective modules of Watch 101 and generate signals corresponding to physiological or environmental conditions. In some implementations, each module or component of Watch 101 may be communicatively coupled via wired or wireless connections, facilitating data exchange and sensor integration. The watch may further incorporate additional or alternative sensors beyond those explicitly described, such as light sensors, oximeters, or other physiological monitoring components.

As illustrated in FIG. 2, Watch 101 is provided solely for illustrative purposes and may include additional or alternative components beyond those depicted. For instance, a variant of Watch 101 may be fabricated with fewer sensors, such as a single temperature sensor 235, while still maintaining functionality for physiological monitoring. In some implementations, a temperature sensor 235 may be separately attached to a user's wrist using a strap or adhesive and wired to an external computing device capable of processing the sensor's data. In other implementations, Watch 101 may include expanded sensor arrays and enhanced processing capabilities to support a broader range of biometric analyses.

The housing of Watch 101 may include multiple structural components designed to enclose and protect internal electronics. The outer housing may serve as a protective shell, while the bottom housing may be designed for direct contact with the user's wrist. The housing may further incorporate insulating layers that electrically isolate internal components from the outer shell, particularly in implementations where the outer housing is fabricated from conductive materials such as metal. The housing may provide structural support for internal components, protecting the device electronics, battery 210, and substrates from mechanical stress, water exposure, and chemical interactions.

The outer housing may be fabricated from various materials, including metals such as titanium, which may provide strength and abrasion resistance while maintaining a lightweight form factor. Alternatively, the outer housing may be composed of polymer materials that offer flexibility and durability. In some implementations, the outer housing may also serve an aesthetic function while maintaining protective integrity.

The bottom housing may be designed to interface directly with the user's skin and may be composed of materials such as medical-grade polymers. In some implementations, the bottom housing may be transparent to allow for the transmission of optical signals, such as those emitted by PPG LEDs. The bottom housing may also be molded onto the outer housing using manufacturing techniques such as injection molding to ensure a secure and ergonomic fit.

The wristband of the wearable device is constructed from body-compatible materials, ensuring safe and prolonged contact with the skin. It is designed to support the integration of Electrodermal Activity (EDA) sensor(s), which are strategically positioned within the wristband and in contact with the user's skin, for monitoring physiological responses, such as skin conductance. The wristband may be further configured to facilitate the transmission of data from the EDA sensors to the watch's electronic components, wherein electrical connections are routed through the wristband itself, maintaining a seamless integration with the watch's overall functionality. The materials used in the construction of the wristband are selected to provide both comfort and durability, while also being aesthetically pleasing. Additionally, the wristband is designed to provide flexibility and wearability, continuous wearability by the user throughout various daily activities and during sleep without compromising the quality of sensor data or user comfort.

Watch 101 may incorporate one or more substrates, which may house the device electronics and power components. These substrates may include printed circuit boards (PCBs), including flexible PCBs made of materials such as polyimide. The substrates may include surface-mounted components using surface-mount technology (SMT), allowing for compact and efficient integration of electronic circuits. Electrical traces within the substrates may facilitate communication between various modules, including power delivery from battery 210 to the device electronics.

The arrangement of device electronics, battery 210, and substrates within Watch 101 may vary depending on implementation. In some aspects, the sensors, including the PPG system 230, temperature sensors 235, and motion sensors 225, may be positioned to interface with the underside of the user's wrist, ensuring optimal data collection. The battery 210 may be positioned separately, such as on an opposing substrate, to optimize weight distribution and internal space efficiency.

The various components and modules of Watch 101 represent functional circuitry designed to facilitate the operations described herein. These modules may include discrete or integrated electronic circuits that implement analog and digital functions. The modules may incorporate amplification circuits, filtering circuits, analog-to-digital conversion circuits, and other signal conditioning elements. Additionally, the modules may include digital processing elements such as combinational logic circuits, memory components, and embedded microcontrollers.

The memory module 202 of Watch 101 may comprise volatile and non-volatile memory, including RAM, ROM, NVRAM, EEPROM, flash memory, or other storage media. The memory module 202 may store various data types, including motion data, temperature readings, and PPG signals collected by the device sensors. Furthermore, the memory module 215 may store instructions that, when executed by one or more processing circuits, facilitate the operation of the watch's various functions.

The functions attributed to the modules of Watch 101 may be implemented using processors, hardware, firmware, software, or a combination thereof. The division of functionality into separate modules is intended for clarity and does not necessarily indicate separate physical components. In some implementations, multiple functional aspects may be integrated within a single hardware or software module.

The processing module 208 of Watch 101 may include one or more processors, microcontrollers, digital signal processors, system-on-chip (SoC) devices, or other processing units. The processing module 208 may facilitate communication between various components of Watch 101, including sensors, memory, and communication modules. In some implementations, the processing module 208 may include integrated features such as Bluetooth Low Energy (BLE) transceivers for wireless communication. The memory module 202 may store computer-readable instructions that, when executed by the processing module 202, enable the functionalities described herein.

This description is provided to illustrate examples of Watch 101 and its various components and does not represent an exhaustive listing of possible implementations. The specific design and component integration may vary based on engineering considerations, user requirements, and advancements in wearable technology.

The communication module 212 within the watch 101 enables seamless data transmission between the watch 101 and the user personal device 105. This module may include circuits for both wireless and wired communication, allowing for interactions between the two devices. In some implementations, the communication module may incorporate wireless technologies such as Bluetooth and/or Wi-Fi, or it can feature wired communication protocols like Universal Serial Bus (USB). The communication module 212 facilitates the exchange of various data types, including motion data, temperature data, pulse waveforms, heart rate data, heart rate variability (HRV) data, photoplethysmogram (PPG) data, and status updates such as charging status, battery level, and configuration settings. Additionally, the processing module 208 of the watch 101 is responsible for transmitting and receiving this data to and from the user personal device 105, and can also receive software or firmware updates from the user device.

Powering the watch 101 is a battery 210, typically a rechargeable Lithium-Ion or Lithium-Polymer type, though alternative battery technologies may also be used. This battery 210 is capable of supporting wireless charging in some implementations. In other cases, the watch may use a capacitor as a power source. The battery or capacitor is designed to fit within the watch's geometry, ensuring a compact and efficient power solution. In addition to the battery, a charger or alternative power source may include sensors capable of collecting supplementary data beyond what the watch 101 itself gathers. This charger or power source may also function as a user personal device 105, handling the storage, processing, and transmission of data between the watch 101 and the server(s) 150.

The power module 204 inside the watch 101 is responsible for managing the charging process of the battery 210. It interfaces with an external wireless charger that replenishes the battery when properly aligned with the watch. The charger is designed to ensure precise alignment and coordination during the charging process. The power module 204 also regulates the voltage, manages the distribution of power to the watch's electronics, and monitors the state of charge of the battery. Furthermore, the battery 210 may include a protection circuit module (PCM) that safeguards against high current discharge, overvoltage during charging, and under voltage during discharge. Electrostatic discharge (ESD) protection is integrated into the power module 204 for added safety.

For environmental monitoring, the watch 101 features one or more temperature sensors 235, which are electrically connected to the processing module 208. These sensors are used to detect the temperature of the user's skin, for example, at the location where the watch makes contact with the user. The temperature data generated by the sensor is sent to the processing module 208, which uses this data to estimate the user's temperature. The temperature sensor may be placed in direct contact with the user's skin, or it could be separated by a thin thermally conductive barrier within the inner housing of the watch. The watch design may include thermally conductive areas that transfer heat from the user's skin to the sensor and thermally insulative areas that help shield the sensor from ambient temperature variations.

The temperature sensor 235 may output a digital signal that the processing module 208 processes to determine the temperature. In some cases, if the sensor is passive, the processing module 208 or a dedicated temperature sensor module may detect changes in current or voltage to infer the temperature. A variety of temperature sensing components may be used, such as thermistors (e.g., negative temperature coefficient thermistors), resistors, transistors, diodes, or other electronic components.

The processing module 208 continuously samples the temperature at regular intervals, such as once per second, though this rate can be configured based on specific needs or power constraints. The watch 101 may sample temperature data throughout the day and night, ensuring enough data for meaningful analysis. This sampled temperature data is stored in memory 202, where the processing module 208 can analyze it to generate average temperature values over defined periods. For example, the average temperature may be computed every minute by summing the individual samples and dividing by the total number of readings. To optimize storage, the memory 202 may store these averages rather than raw data from every sample.

The watch's memory 202 may also store the configured sampling rate, which could vary during the day depending on usage patterns. For example, the sampling rate may increase during periods of physical activity or decrease during sleep. The watch 101 also features filtering capabilities, allowing it to discard unreliable data, such as temperature spikes caused by external factors like hot showers or erroneous readings due to excessive motion detected by the motion sensor 225.

Once the temperature data has been processed and stored, the communication module 212 transmits the sampled and/or averaged temperature data to the user personal device 105. The user device may store this data or process it further before transferring it to a server 150 for long-term storage and more in-depth analysis. The watch 101 features a sophisticated temperature sensing system, incorporating one or more temperature sensors 235 distributed along the inner housing 205-a, near the user's finger. These sensors can function independently or in conjunction with other components such as the accelerometer 225 or the processing module 208. The positioning of these sensors allows the watch 101 to capture accurate, localized temperature readings, offering insights into the user's physiological state.

The processing module 208 is responsible for managing data acquisition from these temperature sensors 235. It processes and stores the individual readings or, alternatively, averages them over time. In some cases, the processing module 208 may compute a single representative temperature value by integrating data from multiple sensors placed at different locations on the user's finger. This allows for a more comprehensive and reliable temperature assessment, particularly in areas where localized temperature fluctuations may occur.

Temperature sensors 235 are capable of detecting distal temperature changes in various parts of the user's finger, such as the underside, which is more sensitive to environmental and physiological factors. Continuous temperature monitoring at the finger can reveal subtle fluctuations that might not be apparent from core temperature measurements alone. These fluctuations can offer unique insights into the user's health, such as identifying early signs of illness or changes in metabolic rate. The processing module 208 dynamically adapts to these variations to provide the most accurate and relevant temperature data.

In addition to temperature monitoring, the watch 101 includes a photoplethysmogram (PPG) system 235, which utilizes optical transmitters and receivers. These optical components work together to emit light onto the user's finger and detect the reflected or transmitted light. The changes in light intensity correlate with variations in blood volume, specifically blood flow changes due to pulse pressure, providing a real-time measurement of the user's cardiovascular health.

The PPG system 235 can be configured as either a reflective system, where light is reflected back to a receiver, or a transmissive system, where light passes through the finger to a receiver on the opposite side. The configuration of the optical transmitters and receivers may vary depending on the specific design and intended use of the system. Typically, optical transmitters, such as LEDs, emit light in the infrared or other spectrums, while receivers like photosensors, phototransistors, and photodiodes detect the reflected or transmitted light, generating a PPG signal.

To optimize the accuracy of the PPG measurements, the processing module 208 controls the transmitters and samples the PPG signal at a defined rate (e.g., 250 Hz). The system dynamically selects the transmitter that provides the strongest signal for continuous light emission, ensuring consistent and reliable data capture.

The pulse waveform produced by the PPG system 235 reflects blood pressure changes across multiple cardiac cycles. This waveform may also indicate respiratory-induced variations, which can be analyzed to determine the user's respiratory rate. The processing module 208 stores this pulse waveform in memory 202 for further analysis, contributing to a comprehensive physiological profile of the user.

From the pulse waveform, the processing module 208 may calculate heart rate by analyzing the interbeat intervals (IBI), which measure the time between successive heartbeats. These IBI values, along with the computed heart rate, are stored in memory 202 for tracking and analysis over time. In the same way, other relevant sleep related factors may be extracted from the PPG signal(s) and either transmitted to a gateway device 105 in raw or preprocessed format for further analysis, or used by a sleep model that is executed on the wearable device.

Heart rate variability (HRV) is also derived from variations in the interbeat intervals. HRV is an important indicator of the user's autonomic nervous system and overall health. The processing module 208 computes HRV by analyzing these IBI variations, and the resulting HRV values are stored in memory 202 for further study. In addition, the algorithm used to calculate HRV is dynamic and may be updated over time, ensuring that the analysis remains accurate and relevant as the system improves and adapts to user-specific data patterns.

Similarly, the watch 101 supports continuous monitoring of SpO2 (blood oxygen saturation). SpO2 is measured by using the PPG system 235 to analyze changes in light absorption as it passes through the user's skin, specifically assessing the amount of oxygenated blood in circulation. The processing module 208 uses this data to calculate the user's SpO2 levels. Just like HRV, the SpO2 algorithm is dynamic, meaning that it can evolve and improve over time based on new data inputs and advancements in algorithmic processing.

The watch 101 continuously collects and processes this data, with memory 202 storing heart rate, HRV, SpO2, and other physiological metrics for analysis. The processing module 208 uses these stored values to track trends over time, providing the user with valuable insights into their health and wellness. Additionally, the watch 101 can adapt to changing conditions, ensuring that measurements like SpO2, HRV, and heart rate remain accurate even as the user's behavior or health status evolves.

The wearable device, watch 101, may include one or more event buttons (220). These event buttons can range from a simple single push button to more advanced versions equipped with programmable logic to accommodate various user inputs. Depending on the user's actions, these inputs may trigger different operating modes of the watch 101.

The signals from the event button(s) may be transmitted continuously, along with other physiological data or their transformed versions. In some implementations, the event button signals could be transmitted periodically or at specific intervals, often accompanied by a timestamp to mark the exact moment of the event.

To communicate different operational states, the watch 101 may be equipped with one or more LED light indicators. These LED lights may be used to signal various events or statuses, providing a simple yet effective visual output. For example, the LED lights could display specific colors or blink patterns to indicate different functionalities, such as the progress of signal acquisition, whether the model is running in an online or offline mode, or the status of data transmission. Additionally, the LEDs may serve as a visual alert system, warning the user if a potential health issue is detected or if an alarming event has already occurred.

In some implementations, the watch 101 may also include one or more Electrodermal Activity (EDA) sensors. These sensors may be integrated into the watch's wristband, targeting different locations around the user's wrist, or placed on the body of the watch, where they come into direct contact with the user's skin. EDA sensors measure the skin's electrical conductance, which may change in response to stress or emotional states, providing valuable insights into the user's physiological condition.

The watch 101 may include one or more motion sensors 225, such as 3-axis accelerometers and/or gyroscopes (gyros), which generate signals indicating the movement of the sensors. For example, the watch 101 may include accelerometers that generate acceleration signals to reflect the device's acceleration. Similarly, the watch 101 may have gyro sensors that produce gyro signals to track angular motion, such as angular velocity or changes in orientation. These motion sensors 225 may be part of one or more sensor packages. One example of a sensor used in the watch 101 is the Kionix KXG03 inertial micro-electromechanical system (MEMS) sensor, which combines a 3-axis accelerometer and a 3-axis gyroscope to measure angular rates and accelerations along three perpendicular axes.

The processing module 208 samples the motion signals at specific rates, such as 50 Hz, to determine the motion of the watch 101 based on the sampled data. For instance, the processing module 208 may sample acceleration signals to calculate the watch's acceleration or gyro signals to track angular motion. It may also store motion data in memory 202, including both raw sampled data and calculated motion data such as acceleration and angular values derived from the sampled signals.

The watch 101 is capable of storing various data types, such as temperature data (both raw sampled and calculated averages), PPG signal data (e.g., pulse waveforms and derived data such as heart rate, interbeat interval (IBI) values, HRV, and respiratory rate), and motion data (e.g., sampled motion data indicating both linear and angular motion). The watch 101, or other connected devices, may calculate and store additional values derived from the physiological data it collects. These derived values may include sleep scores, activity levels, readiness metrics, and more.

For motion data, derived values could include motion counts, regularity, intensity values, metabolic equivalent of task (METs), and orientation values. Motion counts, regularity, intensity, and METs measure the amount and intensity of user movement (e.g., acceleration and velocity) over time. Orientation values provide information on how the watch 101 is positioned on the user's finger, such as whether it is worn on the left or right hand.

In some implementations, motion counts and regularity values are determined by counting the number of acceleration peaks within specific time intervals, like 30 seconds to 1 minute. Intensity values quantify the number and intensity of movements, categorized as low, medium, or high, based on the acceleration thresholds. METs are calculated based on movement intensity, movement regularity, and movement frequency within a given period, such as 30 seconds.

To optimize storage space, the processing module 208 may compress the data stored in memory 202. For instance, after calculating metrics from the sampled data, the processing module 208 may delete or average the raw data over longer periods. If the watch stores average temperatures every minute, it may calculate five-minute averages and delete the one-minute averages to free up storage space. Data compression depends on factors like the available memory 202 and the time elapsed since the last data transmission to the user personal device 105.

While the watch 101 is equipped with sensors for measuring physiological parameters, other devices can also collect similar data. For example, although the watch 101 can measure temperature using its built-in temperature sensor 235, other devices, such as wrist-based wearables or medical devices, can also capture these physiological parameters. These sensors may include wearable medical devices, implantable devices, or other computing devices designed to capture physiological signals.

Physiological measurements may be taken continuously throughout the day and night. However, in some cases, the watch 101 may measure parameters during specific times, such as when the user is resting, active, or sleeping. For example, the watch 101 may acquire more accurate physiological data when the user is at rest or asleep. In such cases, the watch can detect when the user is in a resting or sleeping state and collect more precise physiological parameters like temperature. This data can be used alongside data collected during other states to provide a comprehensive understanding of the user's health.

The watch 101 may also be equipped with additional physiological sensors that allow for more complex analysis. These sensors may measure other parameters, such as blood oxygen levels, heart rate variability (HRV), or other vital signs. The processing module 208 may continuously process the sensor signals over time, transmitting meaningful intermittent physiological data that can be used in health models. The data may be transmitted in raw or processed formats, providing valuable insights into the user's health. This data, whether raw or processed, can be further analyzed by the user personal device 105 or a server 150, supporting ongoing health monitoring and personalized health recommendations.

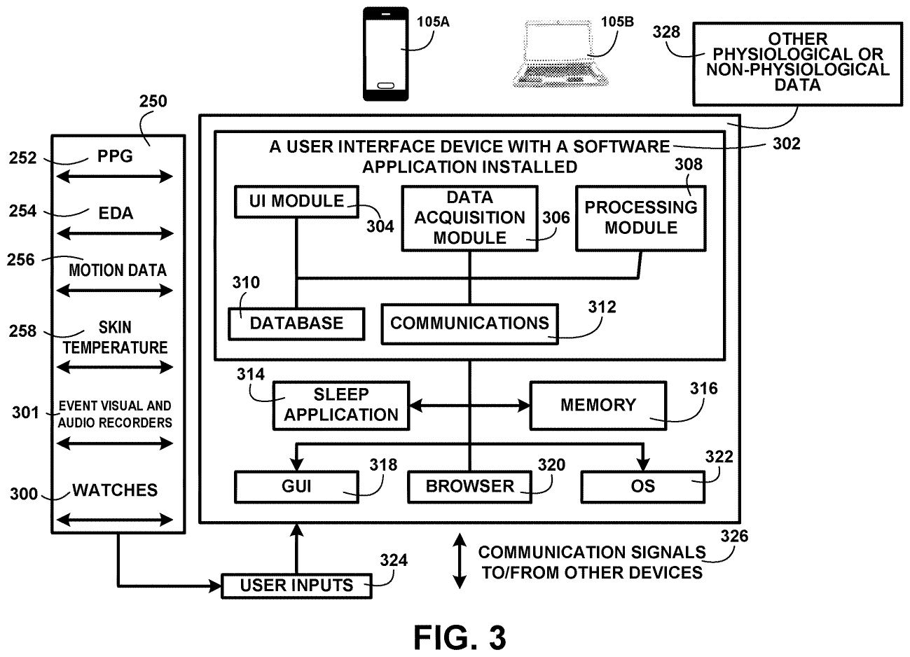

The system 100 further includes a user personal device 105, such as a smartphone, which is in communication with the watch 101. This communication may be either wireless or wired, depending on the implementation. The watch 101 sends various types of data to the user personal device 105, including but not limited to temperature data, photoplethysmogram (PPG) data, motion/accelerometer data, and the wearable 101 input data. Additionally, the user personal device 105 may send data back to the watch 101, such as firmware or configuration updates to ensure that the watch operates optimally. FIG. 3 shows for illustrative purposes only an example of a user device that communicates with one or more wearable devices, as well as other possible sensor and non-sensor devices and/or medical databases, all of which support the algorithm(s) and methodology of one embodiment.

In certain embodiments, each user device comprises one or more hardware modules including a memory module, a processing module, a power module, a battery module, a communication module, and one or more sensor modules. The memory module may store executable instructions, sensor data, processed data, and model parameters. The processing module may comprise one or more microprocessors, microcontrollers, or other computing units configured to execute instructions for data acquisition, signal processing, feature extraction, and sleep stage classification.

In certain embodiments, the sensor modules include one or more physiological sensors configured to acquire signals indicative of a user's physiological state. Such sensors may include photoplethysmography (PPG) sensors for measuring blood volume changes, electrodermal activity (EDA) sensors for measuring skin conductance, temperature sensors for measuring skin temperature, and motion sensors such as accelerometers for detecting user movement. In certain embodiments, EDA sensors may include electrodes configured to contact the user's skin, while PPG sensors may include optical emitters and detectors configured to measure reflected or transmitted light.

In certain embodiments, the device further comprises one or more user input components, including event buttons configured to allow a user to input event signals, and one or more user interface elements, including displays, indicators, or haptic feedback mechanisms. The communication module may be configured to transmit data to and receive data from external devices or server systems using wireless communication protocols. The power module and battery module may provide electrical power to the device and may support continuous or intermittent operation during sleep monitoring periods.

FIG. 3 shows for illustrative purposes only an example of a user interface device with a software application installed configured to receive data, process data, store data, and display data associated with sleep staging and physiological monitoring, wherein the user interface device with a software application installed includes a UI module 302, a database 304, a data acquisition module 306, a processing module 308, a communications module 310, a sleep application 312, a GUI 314, communication signals to/from other devices 316, memory 202, and browser OS 320 mem, and the data acquisition module 306 is configured to receive data including PPG 252, EDA 254, motion data 256, skin temperature 258, event visual and audio recorders 301, watches 300, and other physiological or non-physiological data 328, and the processing module 308 and sleep application 312 are configured to process the received data and generate sleep staging data and other physiological analysis data for display on the GUI 314 and storage in the database 304 and memory 202, and the communications module 310 is configured to transmit and receive communication signals to/from other devices 316.

FIG. 3 displays the block diagram for user device(s) 105 that act as gateways, connecting to various devices that provide data essential for running sleep algorithm(s). The user device(s) serve as the central hub for collecting, processing, and transmitting the physiological data necessary to analyze sleep patterns. These devices may be connected to additional computing resources via a network, allowing for more complex computations if necessary.

The user device(s) may communicate directly with the wearable device (component 250), controlling the wearable's firmware and managing the flow of collected physiological signals. In some implementations, the user personal device 105 may only receive the specific signals from the peripheral devices that are required for running the sleep algorithm, ensuring that only the relevant data is transmitted. This selective communication helps optimize the system by conserving energy and extending the battery life of the wearable device, making it more efficient for long-term use.

The user personal device 105 includes various applications, including a software application, to process the transmitted data from various devices. The user device may be capable of running an entire sleep algorithm to produce results in real time or retrospectively at the end or during sleep session(s). The user personal device 105 may include an operating system (OS) 325, a web browser (e.g., web browser 322), and a graphical user interface (GUI) 320. The user personal device 105 may also contain other modules and components, such as sensors and audio devices. The wearable application on the user personal device 105 is designed to acquire data from the watch 101 and other wearable and non-wearable devices 250, store it, and process it as needed. For instance, the wearable application may include a user interface module 302, an acquisition module 3005, a processing module 308, a communication module 312, and a storage module (e.g., database 310) for managing application data.

Data processing operations described here may be performed by the watch 101, user personal device 105, servers 110, or a combination of these. For example, data collected by the watch 101 may be pre-processed and transmitted to the user personal device 105, where further processing may occur. Alternatively, data may be sent to the servers 150 for processing, particularly if high processing power is required. In cases where lower processing power or reduced latency is needed, the user personal device 105 may handle those operations before transmitting data to the servers 150. The user personal device 105 also plays a role in processing data. In certain implementations, the user personal device 105 can transmit the collected data to a server 150 for further processing or storage.

This flow of data between the watch, user device, and server enables a seamless experience for tracking and analyzing various physiological metrics over time. Through this system, the watch 101 can continuously monitor and evaluate the user's health data, providing insights that are useful for maintaining and improving overall well-being. The user personal device 105 serves as the gateway to connect with the wearable device(s). It collects all sensory data and relevant information from the wearable, which is necessary for detecting and analyzing sleep stages. This data includes physiological signals like temperature, heart rate, and motion, which help the system evaluate the user's sleep patterns accurately.

The user personal device 105 can connect to either a single wearable watch 101 or multiple wearable devices. This flexibility allows users to use different wearables or to expand their monitoring system to include multiple devices, depending on their needs. The ability to connect with several devices ensures that the system can gather comprehensive data from various sources to enhance sleep tracking and health assessment.

In some implementations, the user personal device 105 can also connect to edge sensor data from other devices (340) that monitor the sleep environment. For example, devices such as ambient temperature sensors, humidity monitors, or noise detectors can provide additional contextual data to better understand the user's sleep conditions. This external data, combined with the wearable physiological data, improves the accuracy of sleep stage detection.

Users can also input additional information about their health status and daily activities into the user personal device 105. A short questionnaire may be used to provide the sleep detection algorithm with more contextual information. This helps the algorithm refine its predictions by accounting for factors such as exercise, stress, or other lifestyle variables that could influence sleep quality.

In some implementations, the user personal device 105 may handle the entire sleep algorithm, depending on the complexity of the algorithm and the device's processing power and memory resources (310, 318). If the device has sufficient resources, it can run the sleep detection model entirely on the user device, providing near-instant feedback on sleep patterns and other health metrics.

Alternatively, in some setups, the wearable watch 101 may handle some of the data transformation and signal preprocessing (using processing unit 202). The user personal device 105 may then take over for the model inference (via running an sleep application 315) or even handle the entire prediction process. This division of processing power between the wearable and the user device helps optimize processing efficiency in real-time or near-real-time sleep stages inferences.

In some implementations, the user device (105) is equipped with memory 318, which serves several important functions. This memory can store the results of inferences or predictions made by the sleep algorithms based on the physiological data collected. Additionally, the memory may store model parameters that are critical for executing these algorithms, allowing the device to perform sleep analysis even when the connection to the cloud or other remote servers is limited or unavailable.

Furthermore, the memory 318 on the user personal device 105 may also hold historical data about the user's previous sleep patterns, health information, and behavioral data, such as activity levels, entered manually by the user (330) (e.g., through a questionnaire or graphical interface 320). This historical data can be used to personalize the sleep algorithm, providing more accurate and tailored insights about the user's sleep. The user device's memory may not always be confined to local storage. In some implementations, the memory may also be remotely located, such as in cloud storage, and accessible through communication module 312. In such cases, the user personal device 105 can send and receive data via a network system, allowing for seamless synchronization with remote storage. This enables the device to retrieve additional information (e.g., updated models, new data streams, or improved algorithm versions) when needed, while still offering the flexibility to run algorithms locally when the network connection is unavailable or when conserving bandwidth or power is important.

User's device 105 can receive both physiological and non-physiological data from various wearable and non-wearable devices (250, 340), enhancing sleep stage classification. Wearable devices like smartwatches, rings, and fitness trackers provide data on heart rate, HRV, accelerometer readings, and skin temperature. Medical-grade wearables, such as EKG monitors, offer detailed heart activity, while continuous e monitors provide insights into blood pressure variations during sleep. Other examples may include sleep apnea devices that track oxygen levels also contribute to identifying sleep disturbances. Additionally, user input data 330, such as information about daily activities, stress, or medications, helps improve the algorithm's accuracy.

In some implementations, users may be prompted to input text about their feelings and health conditions before going to bed, or they may use the audio prompt system of their personal device to describe their health or answer a questionnaire. Additionally, users may provide information about their daily activities, including their normal or abnormal sleeping patterns, daily activity levels, exercise routines, diet, recent travel, jet lag, depression, anxiety, and other relevant factors. The answers and provided information, expressed in the user's natural language, may then be analyzed using a language model process either on the user's own device 105, on the server(s) 150, or through external language processing services accessible via the network 140. This process allows for the extraction of meaningful insights from the user's input, which can be integrated with other physiological data to enhance the accuracy and personalization of the sleep analysis and predictions. Moreover, the system may employ a circadian analysis model in conjunction with other traditional or state-of-the-art models to interpret all of the provided information when determining sleep stage classifications, confidence levels, and during the post-processing of sleep stage transitions. This approach ensures a comprehensive evaluation of the user's sleep patterns, accounting for both internal biological rhythms and external influencing factors.

In the absence of an event button, users may still be able to input information about their sleep session using the graphical user interface of the sleep application 320 on their device. This interface allows users to manually log additional details, such as the time they went to bed or woke up, or any relevant sleep disturbances, further enhancing the accuracy of the sleep detection system.

The watch 101, user personal device 105, and server 150 within the system 100 are designed to assess sleep patterns for the user by collecting and analyzing data. The system can use data gathered from the watch 101, such as temperature, heart rate, HRV, and other metrics, to generate scores like Sleep Scores or Readiness Scores. These scores are calculated based on data collected during sleep periods and may be associated with specific “sleep days,” which could be adjusted to reflect the user's personal sleep schedule. For instance, sleep days might align with traditional calendar days, running from midnight to midnight, or they could be offset based on the user's typical sleep cycle. This flexibility allows the system to evaluate sleep patterns in a manner that matches individual sleep schedules, with users potentially adjusting the sleep day timing via the device's interface.

Each score, whether it's a Sleep Score or sleep Readiness Score, is derived from various contributing factors. A Sleep Readiness Score is a metric used to evaluate how prepared a person is for sleep based on various physiological, behavioral, and environmental factors. It provides an indication of how likely a person is to fall asleep easily and experience high-quality sleep. The score may be calculated using data gathered from the wearable watch device 101.

For the Sleep Score, contributors might include total sleep duration, sleep efficiency, restfulness, REM sleep, deep sleep, latency, and sleep timing. Total sleep refers to the sum of all sleep periods within the sleep day, while sleep efficiency is calculated by comparing the time spent asleep to the time spent awake in bed. Restfulness indicates the quality of sleep and may be based on wake-up counts, excessive movement, and instances of the user getting up. REM and deep sleep are calculated as the total duration spent in those stages, and latency measures the time it takes for the user to fall asleep. The timing contributor reflects the relative timing of sleep periods, which can be weighted based on their duration.

In addition to Sleep Scores, a sleep Readiness Score is also calculated based on multiple contributors, including sleep quality, heart rate, HRV balance, activity levels, and temperature. Sleep balance reflects how consistent the user's sleep duration is over time, while resting heart rate and HRV balance provide insight into recovery and overall health. The recovery index measures the stabilization of the resting heart rate during sleep, indicating the body's ability to recover for the next day. Body temperature is monitored to assess any significant variations from the user's baseline, which could highlight potential health issues.

The system 200 also may support parts or all parts of sleep stages classification model(s) using data from accelerometers, PPG, and autonomic nervous system signals. This data allows for multi-stage sleep detection, offering a more detailed analysis of the user's sleep cycle.

Wearable devices that track sleep have become increasingly popular as people recognize the importance of sleep for overall health, including physical health such as weight management, immune function, and blood-sugar regulation, as well as mental health, including cognitive function, mood, and stress levels. These devices offer users a daily feedback loop to help them understand their sleep patterns and make behavioral changes that could improve their healthspan and lifespan. The effectiveness of these devices depends not only on the form factor but also on the accuracy of the data they provide, as users are more likely to adopt and stick with devices that deliver meaningful, real-world insights.

Moreover, there is growing interest in how sleep tracking data from wearable devices can be utilized by researchers and clinicians. The goal is to understand how accurate these devices are when compared to traditional, gold-standard methods of sleep measurement, such as polysomnography (PSG). PSG is a comprehensive test that records multiple physiological signals, including EEG, EOG, ECG, EMG, and sometimes PPG, to stage sleep and identify different stages such as N1 (light sleep), N2 (light sleep), N3 (deep sleep), REM, and wakefulness. This detailed data is typically used by experts or algorithms to determine sleep stages, and the process is crucial for understanding sleep disorders. Research into the accuracy of wearable devices relative to PSG can help integrate these devices into both clinical and research settings, offering the potential for large-scale healthcare management.

In sleep staging, PSG typically uses 30-second segments, with the overall inter-scorer reliability reported to be 82-83%. However, reliability tends to be weakest for the N1 stage, which is a transitional phase between wakefulness and sleep. In wearable devices, N1 sleep is often combined with N2 sleep, and this combined stage is referred to as light sleep. Understanding these stages and improving the accuracy of sleep tracking with wearables could have significant implications for both individual health management and clinical research.

In addition to traditional polysomnography (PSG), actigraphy has been used to monitor a user's sleep/activity cycles and assess sleep-wake patterns. However, actigraphy has limitations in quantifying certain features of sleep, particularly in the accurate classification of sleep stages. When compared to PSG, actigraphy typically exhibits a sensitivity range of 72-97% and specificity range of 28-67%, with Pearson's correlation coefficients for metrics like total sleep time (TST), sleep onset latency (SOL), and wake after sleep onset (WASO) varying between 0.36-0.97. Although actigraphy can provide useful insights for basic wake-sleep assessment, it struggles with differentiating non-rapid eye movement (NREM) and rapid eye movement (REM) sleep stages, making it less reliable for more detailed sleep quality analysis.

When combined with measures from the autonomic nervous system (ANS), actigraphy may significantly improve sleep quality estimations. The integration of ANS data, typically acquired via wearable devices, enhances the accuracy of sleep-wake assessments, bringing it on par with consumer EEG devices. For example, adding ANS features to actigraphy can improve Cohen's kappa from 0.5 to 0.6, showing an improvement in sleep stage classification. This combination leverages the strengths of accelerometer data, ANS data, and machine learning techniques, such as multidimensional sensor streams, to classify sleep stages with greater precision, achieving four-class sleep stage classification (e.g., awake, light sleep, REM sleep, deep sleep).

Despite advancements, conventional wearable devices still face several challenges when it comes to sleep detection and stage classification. One major issue is the limited amount of sleep data collected and analyzed in real-world settings, which affects the generalizability of results. Additionally, there has been insufficient exploration of how different sensor data, such as ANS signals and circadian models, contribute to sleep quality evaluations across diverse populations. Moreover, lower quality ANS data, often collected from peripheral body parts like the wrist or arm, introduces significant noise, distorting the accuracy of sleep assessments. Furthermore, while accelerometer, ANS, and circadian rhythm data can reveal physiological changes during sleep, no comprehensive study has analyzed the relative impact of these features across large, diverse datasets. Additionally, complex machine learning models, which are effective offline, struggle to perform well on wearable devices due to their computational limitations and the difficulty in integrating multiple models for real-time predictions.

Automatic sleep stage classification has long been a challenge, primarily due to the reliance on subjective interpretation by human annotators and imperfect reference data. This results in discrepancies between different studies and challenges in developing reliable, real-time wearable solutions. Many conventional devices only accurately detect a subset of sleep stages, typically two or three, leading to inaccurate or incomplete sleep assessments. As a result, wearables have yet to achieve consistent and reliable classification of all sleep stages across a variety of users.

The system may support automatic sleep stage classification by utilizing data from wearable devices to determine when a user is awake or in specific sleep stages, such as light sleep, REM sleep, or deep sleep. This classification can then be presented to the user through the device's graphical user interface (GUI). By leveraging these techniques, the system offers a more comprehensive understanding of a user's sleep patterns, allowing individuals to adjust their habits to improve overall sleep quality and health.

For example, the wearable watch can continuously monitor physiological data from the user throughout the night. Using advanced sensors, such as LEDs (green, red, and IR), the watch collects data from arterial blood flow in the user's wrist. This data may include heart rate, accelerometer, temperature, and blood oxygen levels, among others. The use of multiple LED types enhances performance in varying conditions, such as during physical activity, where green LEDs provide superior performance compared to other light sources. Additionally, by placing multiple LEDs around the watch, the system can improve signal acquisition, especially compared to devices like rings or other wearables where LEDs are often positioned too closely together.

The watch's accelerometer can record motion data at a sampling rate of 50 Hz to track movements during sleep. This data can be processed to calculate key metrics such as maximum, minimum, and mean accelerometer values, which provide insights into the user's sleep patterns. The watch may apply a 5th order Butterworth bandpass filter to the raw data to remove noise and refine motion analysis. In addition, the system can calculate the mean amplitude deviation (MAD) of the accelerometer's vector magnitude, which is useful for analyzing activity levels during sleep.

Temperature data is another crucial factor for sleep detection, and the watch includes NTC thermistors to measure skin temperature at regular intervals. The temperature is aggregated into 30-second epochs, ensuring that it aligns with other sleep metrics. These temperature readings are analyzed to identify physiological changes, such as fluctuations in core body temperature, which can indicate sleep onset or specific sleep stages. A key pattern observed is that finger temperature decreases before sleep onset and rises during NREM sleep, making it a reliable marker for determining sleep onset times. In addition to temperature, the system also processes raw photoplethysmogram (PPG) data to assess heart rate and heart rate variability (HRV). The PPG system within the watch operates at a sampling rate of 50 Hz, utilizing infrared light to detect beat-to-beat intervals. By applying moving average filters and median filters, the system can identify normal heartbeats and discard anomalies caused by motion or other artifacts. This HRV data is then used to derive various metrics, such as rMSSD, SDNN, and breathing rate, which are valuable for evaluating the user's autonomic nervous system activity during sleep. These HRV features, particularly those in the high-frequency band, are linked to vagal activity, providing further insights into the user's sleep quality and overall health.

Compared to other wearable devices like rings, the watch offers distinct advantages in sleep detection. Its form factor allows for the placement of multiple sensors in a more optimal configuration, which enhances signal acquisition. Additionally, the watch can support more advanced algorithms and processing power, making it better suited for complex tasks like real-time sleep stage classification. With its ability to collect a wide array of data (e.g., heart rate, accelerometer, temperature, PPG) and process that data on the device or transmit it to a connected user device for further analysis, the watch stands out as a comprehensive tool for sleep monitoring and health assessment. The integration of these data points allows for more accurate and reliable sleep analysis, ultimately helping users optimize their sleep quality and overall well-being.

In addition to heart rate, accelerometer, temperature, and photoplethysmogram (PPG) data, other valuable features can be leveraged to improve sleep detection accuracy. For instance, skin conductance or galvanic skin response (GSR) can offer insights into sympathetic nervous system activity, which may vary across different sleep stages. Studies have shown that sympathetic activity tends to decrease during NREM sleep and increase during REM sleep, which makes GSR a useful indicator. Furthermore, respiratory rate can also play a critical role in sleep staging, particularly in detecting apneas or other sleep disorders. The integration of these features, combined with machine learning algorithms, can allow for the differentiation of sleep stages, such as the detection of light versus deep sleep or REM sleep, which may otherwise be indistinguishable based solely on motion and heart rate data.

Moreover, ambient environment data such as light intensity, temperature, and humidity levels in the user's bedroom could be useful for assessing external factors that affect sleep quality. For example, high ambient light levels may interfere with sleep onset, while temperature changes might be correlated with specific sleep stages. Collecting and integrating data from environmental sensors can therefore improve the accuracy of the sleep detection system by accounting for factors outside the user's physiological signals.

Anomalous data can significantly degrade the performance of sleep detection algorithms. These anomalies often arise due to motion artifacts, poor sensor contact, or external interferences, leading to erroneous readings. For instance, during restless sleep or excessive movement, accelerometer data might produce spurious signals that could confuse the algorithm into misclassifying sleep stages. Similarly, PPG data may suffer from noise when the user moves or the sensor becomes dislodged, resulting in unreliable heart rate measurements. To handle these anomalies, the system can use advanced filtering techniques to detect and discard outliers. For example, accelerometer data can be preprocessed by applying a moving average filter to smooth the raw signal and remove abrupt, unnatural spikes caused by motion. If accelerometer readings exceed a certain threshold indicating excessive movement (e.g., large variations in the signal within a short period), the data can be flagged as unreliable for sleep stage classification. Additionally, PPG data can be analyzed using a median filter to identify and remove abnormal heartbeats caused by artifacts. The quality of the PPG signal can also be assessed in real-time, and if the signal quality falls below a predefined threshold, the system can reduce the likelihood of correct sleep stage classification and flag this period as uncertain.

An interesting approach to quantifying the likelihood of correctness is adjusting the classification score (or confidence level) based on the quality of the data. For example, during periods of high motion or low-quality signal, the algorithm could adjust the classification likelihood downward, reflecting the increased uncertainty. This way, the user is presented with more reliable sleep stage classifications, while periods of uncertainty are highlighted for further review or reconsideration.

Motion artifacts are a common source of error in wearable devices used for sleep monitoring. As users move, roll over, or shift during sleep, accelerometer-based devices often misinterpret these movements as significant physiological changes, such as waking or transitioning between sleep stages. These artifacts can lead to inaccurate classifications, such as false awakenings or incorrectly labeling light sleep as deep sleep.

To mitigate these motion artifacts, sophisticated signal processing techniques can be employed. For instance, accelerometer data can be filtered using a bandpass filter that only allows frequencies associated with typical human movements during sleep to pass through. Additionally, machine learning models can be trained to distinguish between sleep-related motion (e.g., body repositioning) and wake-related motion (e.g., turning over to wake up). A model trained on labeled data can use features like the amplitude and frequency of movement to distinguish between legitimate sleep-stage transitions and motion artifacts, reducing the impact of false positives.

Furthermore, combining accelerometer data with other physiological signals, such as heart rate or skin temperature, can improve classification robustness. For example, if an accelerometer detects significant motion, but the heart rate and skin temperature data remain stable, the system may interpret the motion as a sleep position shift rather than waking, maintaining the sleep classification despite motion artifacts.

Skin temperature is another key physiological parameter that changes during the sleep cycle, and its fluctuations can offer important insights into sleep staging. Typically, skin temperature decreases during NREM sleep and increases during REM sleep, corresponding with changes in the body's core temperature. However, skin temperature can also be influenced by external factors, such as the ambient temperature of the room, the use of blankets, or the user's clothing. These environmental variables can distort the physiological signal, causing skin temperature readings to fluctuate erratically and potentially impacting sleep stage classification.

In cases where skin temperature data appears anomalous, it's important to consider both the internal and external factors influencing the data. In some implementations, the system may include checks for extreme values (e.g., skin temperature readings outside a normal range of 31-40° C.) and correct for these anomalies by filtering out outlier data. Moreover, temperature data may be analyzed over time in the context of other physiological signals. For instance, if skin temperature rises rapidly but the user's heart rate and motion remain stable, this may be indicative of external influences (e.g., a blanket) rather than a change in sleep stage. Conversely, a sudden drop in temperature with no corresponding change in other physiological data may indicate a sensor malfunction or artifact that should be discarded.

To address these challenges, the system may leverage contextual information from environmental sensors (e.g., room temperature and humidity) to adjust or normalize skin temperature readings. Multi-sensor approach and considering environmental context, may allow the system to more effectively deal with anomalous data, ensuring reliable sleep detection even in the presence of external disturbances.

In certain implementations, the system is designed to compute autonomic nervous system (ANS)-derived features, such as heart rate and heart rate variability (HRV), using raw photoplethysmogram (PPG) data collected by the watch 101, the user personal device 105, and/or servers 150. The PPG data, collected via the PPG system 230 at 25 Hz at at least one wavelength (for example infrared 900 nm), is gathered primarily during the night (or sleeping time when tracked by the user prompt or triggering the event button) to monitor physiological signals while the user sleeps. The raw PPG data undergoes processing to derive beat-to-beat data, which is essential for computing HRV. A real-time moving average filter is applied to the PPG data to identify the local maximum and minimum values, which mark the timing of each heartbeat. These intervals, or heartbeats, are then examined for abnormalities. For example, if an interval deviates by more than 16 beats per minute (bpm) from the median value, the interval is labeled as abnormal and discarded. Only intervals that are validated as normal (i.e., five consecutive intervals) are kept for further analysis.

Once the high-quality intervals are identified, time and frequency domain HRV features are extracted. These features may include heart rate, root mean square of successive differences (rMSSD), standard deviation of normal-to-normal intervals (SDNN), percentage of successive intervals greater than 50 ms (pNN 50), frequency power in the low-frequency (LF) and high-frequency (HF) bands, total power, normalized power, breathing rate (respiratory rate), and others. These spectral divisions are crucial because they represent different physiological mechanisms. For instance, the HF band (0.15 Hz to 0.4 Hz) is often associated with vagal activity, while the LF band (0.04 Hz to 0.15 Hz) contains spectral power that reflects both sympathetic and parasympathetic influences, though the precise physiological interpretation of the LF band is still debated. Some implementations may also involve calculating the mean and coefficient of variation of the zero-crossing interval, as well as additional nonlinear and statistical features such as Poincaré plot descriptors (SD1 and SD2), sample entropy, approximate entropy, detrended fluctuation analysis (DFA), heart rate fragmentation indices, and higher-order moments like skewness and kurtosis of RR intervals. Wavelet transform features can be used to capture transient patterns, while recurrence quantification analysis (RQA) and multifractal detrended fluctuation analysis (MF-DFA) provide insights into signal complexity and self-similarity. Other potential features include the HRV triangular index (HRVTI), acceleration and deceleration capacities, very-low-frequency (VLF) power, ultra-low-frequency (ULF) power, and spectral entropy, all of which contribute to a more comprehensive assessment of autonomic nervous system dynamics.

In certain embodiments, the system includes a user interface device configured to execute a software application for sleep monitoring and analysis. The user interface device may include a mobile device, tablet, laptop, or other computing device capable of executing application software and presenting graphical user interfaces (GUIs). The software application may include one or more modules, including a data acquisition module, a processing module, a communication module, and a user interface module.

In certain embodiments, the data acquisition module is configured to receive physiological and non-physiological data from one or more wearable devices, external sensors, or remote systems. The processing module may perform local analysis, including feature extraction and preliminary sleep stage classification. The communication module may facilitate data exchange with remote server systems. The user interface module may generate visual representations of sleep data, including sleep stage timelines, sleep summaries, and quality metrics.

In certain embodiments, the graphical user interface may display sleep stage distributions, including percentages of REM, light sleep, deep sleep, and wake states, as well as temporal visualizations of sleep stages over time. The interface may also display confidence scores, sleep quality indicators, and derived metrics such as total sleep time, sleep efficiency, and number of awakenings. In certain embodiments, the interface may provide user feedback, recommendations, or alerts based on detected sleep patterns or anomalies.

In certain embodiments, the system comprises one or more processing subsystems including one or more processors, graphics processing units (GPUs), tensor processing units (TPUs), digital signal processors (DSPs), neural processing units (NPUs), or other specialized processing circuitry configured to execute machine-readable instructions stored in one or more memory devices. The memory devices may include volatile memory, non-volatile memory, flash memory, solid-state storage, or other storage media configured to store executable instructions, intermediate data, feature representations, trained model parameters, and historical datasets. The processing subsystems may be configured to perform operations including data acquisition, signal preprocessing, feature generation, data transformation, model execution, inference, decision-making, and output generation.

In certain embodiments, the system further comprises one or more data storage systems or databases configured to store structured and unstructured data, including raw sensor data, processed data, feature vectors, model outputs, metadata, user-specific data, and system-level data. The databases may be implemented using relational databases, non-relational databases, distributed databases, data lakes, or other storage architectures, and may support indexing, querying, and retrieval operations to facilitate efficient data processing.

In certain embodiments, the system includes one or more artificial intelligence or machine learning components configured to process structured data and generate output data. The machine learning components may include one or more trained models implemented using a variety of model architectures, including but not limited to linear regression models, logistic regression models, decision tree models, random forest models, gradient boosting models (including XGBoost or similar implementations), support vector machines, Bayesian inference models, probabilistic graphical models, hidden Markov models, k-nearest neighbor models, or ensemble models combining multiple model types.

In certain embodiments, the system may utilize neural network-based architectures, including feedforward neural networks, convolutional neural networks (CNNs) for spatial or image-based processing, recurrent neural networks (RNNs) for sequential data processing, long short-term memory (LSTM) networks, gated recurrent unit (GRU) networks, transformer-based architectures utilizing attention mechanisms, attention-based sequence models, graph neural networks (GNNs), autoencoders and variational autoencoders (VAEs) for representation learning, generative adversarial networks (GANs) for data generation, and hybrid or ensemble architectures combining multiple neural network types. In certain embodiments, models may operate on different data modalities or feature subsets and may be arranged in parallel, sequential, hierarchical, or ensemble configurations to generate intermediate and final outputs.

In certain embodiments, the system may include training and updating mechanisms for machine learning models. Training may be performed using labeled datasets in supervised learning frameworks, unlabeled datasets in unsupervised learning frameworks, partially labeled datasets in semi-supervised learning frameworks, or feedback-driven approaches in reinforcement learning frameworks. Training processes may include data preprocessing, feature engineering, model parameter optimization, loss function minimization, regularization, cross-validation, and performance evaluation.

In certain embodiments, training may be performed locally on a device, remotely on a server or cloud computing platform, or using distributed computing techniques such as federated learning, in which model updates are generated across multiple devices without centralized aggregation of raw data.

In certain embodiments, inference operations may be performed in real time, near real time, or batch processing modes. The system may dynamically allocate computational tasks between local devices and remote computing systems based on latency requirements, computational resource availability, bandwidth constraints, or application-specific requirements. In certain embodiments, edge computing devices may perform initial data processing and feature extraction, while cloud-based systems perform more computationally intensive model execution and analysis.

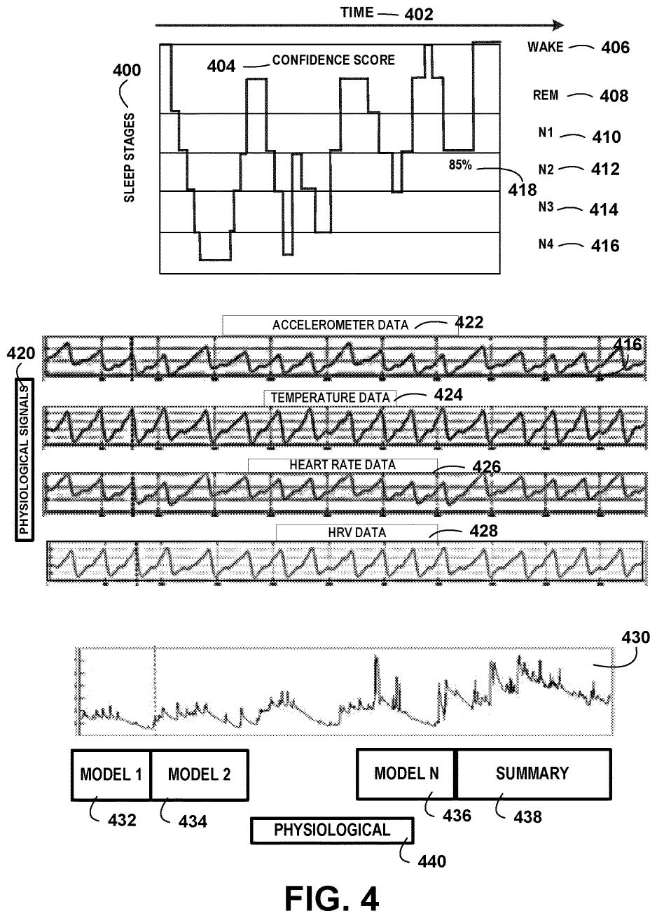

FIG. 4 shows, for illustrative purposes only, an example of graphics displaying how the outputs of a sleep analysis may be presented in a user interface of one embodiment. FIG. 4 illustrates an example of a graphical representation of sleep analysis results presented over time. The graphical representation includes sleep stages 400 plotted across time 402, where sleep stage classifications are displayed along a time axis. A confidence score 404 is shown in association with the sleep stages 400 to indicate a confidence level associated with one or more sleep stage classifications over the time 402. The sleep stages 400 include a plurality of sleep stage classifications including wake 406, REM 408, N1 410, N2 412, N3 414, and N4 416, each corresponding to a sleep stage determined for a time interval or sleep epoch. The graphical representation shows changes in sleep stage classifications across sequential time intervals, and the confidence score 404 represents a confidence value associated with at least one sleep stage classification for a corresponding time interval. The graphical representation may display sleep stage transitions across multiple time intervals to illustrate sleep architecture over a sleep session.

The data acquisition diagram 400, illustrated in FIG. 4, supports sleep staging algorithms by collecting physiological data such as accelerometer data, temperature data, heart rate data, and HRV data. These data streams are continuously collected, without specific time intervals, and can be tagged and color-coded to correspond to the respective sleep stages (awake, light sleep, REM sleep, and deep sleep). This classification of physiological data into sleep stages is integral to the system's ability to make accurate predictions about the user's sleep patterns.

FIG. 4 illustrates an example of the user interface 320, which can visualize and report the collected signal(s) either in diagrams or numerically. The results of the sleep model for a period of classification time may be available for visualization, and such results may be extracted from the system in various formats. Additionally, the results can be stored in a local or remote database for future reference by subsequent models, healthcare practitioners, or users.

Some implementations of the sleep algorithm may classify different stages of sleep over a given sleep period. The number of detected stages depends on the available model(s), the accuracy of the classification, and the nature of the collected physiological data. The sleep algorithms disclosed herein are capable of classifying at least one stage over the course of a sleep session or a sleep epoch.

Certain implementations may calculate a confidence score associated with each classification or classification interval, where the confidence score may vary depending on the model used.

The summary results of all models, or selected models, can be illustrated and reported separately. In some implementations, only the summary may be available; however, the detailed classification results and further analyses may be stored in databases and remain accessible for future use.

In certain embodiments, the system is configured to process physiological signals and generate sleep stage classifications associated with confidence scores. The confidence scores may represent a measure of certainty associated with each classification and may be derived from one or more model outputs, probability distributions, or statistical measures. In certain embodiments, confidence scores may be computed based on model agreement, signal quality, feature consistency, or historical accuracy.

In certain embodiments, the system may generate multiple model outputs for each epoch and may associate each output with a corresponding confidence score. These outputs and confidence scores may be visualized in conjunction with physiological signal traces, including accelerometer data, temperature data, heart rate data, and HRV data. In certain embodiments, the system may utilize confidence scores to adjust or refine sleep stage classifications, including suppressing low-confidence outputs or favoring higher-confidence determinations.

In certain embodiments, the system may further analyze temporal patterns in physiological signals and confidence scores to identify transitions between sleep stages. For example, changes in heart rate variability, motion patterns, or temperature trends may correspond to transitions between REM and non-REM stages. The system may utilize these patterns to enhance classification accuracy and to provide a more robust representation of sleep architecture.

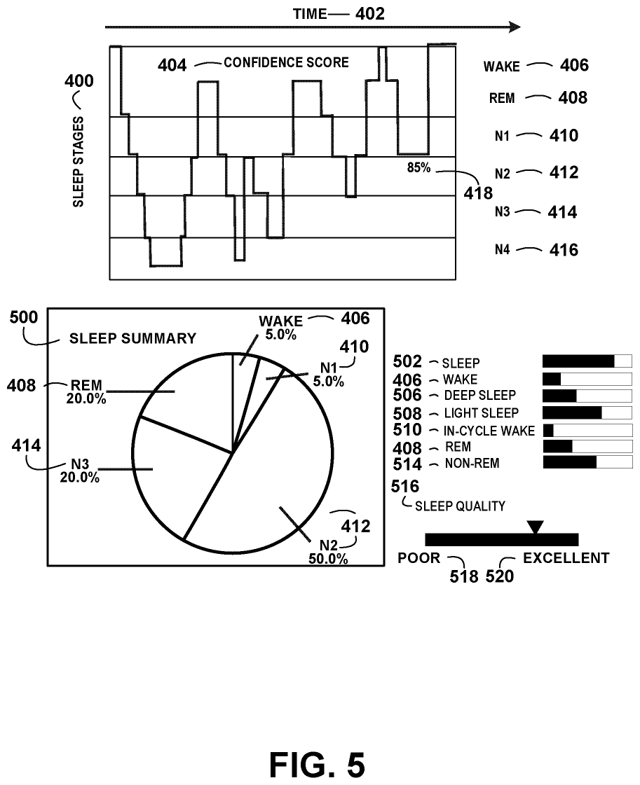

FIG. 5 shows for illustrative purposes only an example of a sleep summary interface presenting statistical information derived from a sleep session of one embodiment. FIG. 5 illustrates a sleep summary 500 displayed over TIME 402, where sleep stage classifications are presented along a time axis together with a confidence score 404. The sleep stage classifications shown in sleep stages 400 include wake 406, REM 408, N1 410, N2 412, N3 414, and N4 416, where the sleep stages are plotted across time 402 to illustrate sleep stage transitions over a sleep period. The sleep summary 500 presents aggregated sleep stage information including stage duration and distribution for one or more sleep stages over the sleep period.

The confidence score 404 represents a confidence value associated with one or more sleep stage classifications across corresponding time intervals. The graphical representation shown in FIG. 5 provides a summary view of sleep stage distribution and sleep stage transitions over the sleep period. FIG. 5 displays a user interface showcasing the sleep summary results for a portion of the classified sleep period. The summary may include key metrics such as the total time of sleep, the duration of deep sleep, light sleep, awakening phases during the sleep cycle, as well as the periods of REM (Rapid Eye Movement) and non-REM sleep. In some implementations, additional data regarding sleep quality, as well as other latent features produced by the models, may also be reported, with or without direct reference to physiological significance.

These latent features could include various factors related to the physiological signals that have been processed and analyzed by the system. In certain implementations, the features extracted from the provided data to generate the model results, whether individually or in combination with reduced dimensionality techniques, may be tabulated and made available for extraction. These features can be reported and stored in databases for future reference, analysis, or clinical use. Furthermore, the results may be visualized either in raw form or after being post-processed, allowing users, practitioners, or systems to interpret the data in different formats.

This flexible reporting and storage system enhances the utility of the data, offering a comprehensive and accessible overview of the sleep analysis while supporting further detailed examination if necessary. In some implementations, the very fine details of the model(s) results, or a combination of model(s) results, can be reported, stored, and visualized. These results may include a comprehensive breakdown of the various factors influencing the outcomes, with the option for detailed, granular analysis.

In some implementations, the results may be ranked according to significance, with the highest confidence level model(s) being highlighted and reported to ensure the most reliable outcomes are prioritized. Furthermore, the dynamic nature of the sensor data and the quality of the collected signals can influence the choice of feature(s) extracted from the signals, or even dictate the selection of a different modeling strategy altogether.

In some implementations, the system may be designed to accommodate such flexibility, allowing for the seamless adaptation of both feature extraction and modeling techniques based on real-time data quality, or overall quality of data collected over a period of sleeping time. All relevant details, including confidence scores, may be presented to users and clinical physicians, providing an additional layer of clarity and precision to facilitate informed decision-making. This ensures that the results can be interpreted with a higher degree of confidence, supporting improved clinical and user-based analysis.

Transformation and preprocessing of the collected physiological data are key aspects in improving the performance of sleep staging algorithms. The transformation may include various types of data calibrations and normalization. The system may apply normalization techniques on a per-night basis or over a period of time, utilizing robust methods like the 5-95 percentiles of the data. This procedure helps account for inter-individual variability, particularly for features like heart rate and HRV, which can vary greatly across individuals due to factors such as genetics and age.

By transforming the physiological data, the system reduces the impact of these inter-individual differences, allowing for more consistent and reliable sleep stage classification. Specifically, HRV features benefit from normalization, as HRV is highly sensitive to sympathetic and parasympathetic activity, which changes across sleep stages. Normalizing these features enhances the system's ability to identify relative changes in HRV over time, improving sleep staging accuracy.

Other engineered features may be used to enhance model performance by tracking overall variability in the data, detecting anomalies, and contributing to the scoring mechanism where the likelihood of predictions is calculated. Additionally, techniques such as z-score normalization, min-max scaling, and adaptive baseline correction may be applied to further refine data consistency and reduce the impact of outliers. While normalization improves the accuracy of the sleep staging algorithm, not all features need to be normalized or transformed.

For example, accelerometer data, which tracks movement, might not be normalized and may instead undergo a specific set of feature engineering processes. The absolute magnitude of movement is particularly essential for detecting periods of awakening or short movements that may indicate an “awake” sleep stage. In such cases, non-normalized accelerometer data can provide valuable information for identifying brief awakenings or disturbances during sleep, ensuring that crucial movement-related patterns are preserved for accurate classification.

To further enhance the classification performance, the system may employ data smoothing techniques using rolling functions, which help account for temporal context by considering both past and future data. This approach mimics how human sleep experts typically score sleep stages-by referencing both the previous and subsequent periods of sleep. Smoothing allows the system to produce more accurate and consistent sleep stage classifications by reducing the effects of random noise and short-term fluctuations in the data.

Furthermore, sliding windows of different lengths (e.g., 30 seconds, 1-minute and 5-minute windows) are used to analyze HRV features, enabling the system to capture both short-term (rapid) and long-term (gradual) changes in parasympathetic activity. These window lengths are chosen based on their correlation with sleep stages, enhancing the system's ability to identify sleep stage transitions.

Beyond sleep staging, the system may also compute sleep-related scores such as the Sleep Score and Readiness Score based on the collected physiological data. These scores provide users with insights into the quality of their sleep and their readiness for the day. Calculating these scores directly on the user personal device 105 helps minimize latency, allowing users to receive real-time feedback. By computing the scores locally on the device, the system avoids potential delays caused by network transmissions to and from the servers 150, enabling faster results. However in some implementations, all or part of such calculations may be carried out on the server(s) 150. The scores may be presented on the user device's GUI 320, allowing users to track their sleep quality and readiness for the day.

In certain embodiments, the system is configured to receive, process, and incorporate event signals generated by a user or detected automatically by the system. Event signals may include user-initiated inputs via buttons, touch interfaces, or voice commands, as well as system-detected events such as abrupt movements, environmental disturbances, or physiological anomalies.

In certain embodiments, event signals may be used to adjust processing operations, including modifying feature extraction parameters, altering model weights, or influencing aggregation logic. For example, a user-indicated awakening event may override model outputs for a corresponding epoch or may be used to recalibrate subsequent classifications.

In certain embodiments, event signals may be stored as part of the user's historical dataset and may be used to identify recurring patterns, triggers, or correlations with physiological data. The system may further utilize such information to refine models, improve prediction accuracy, and provide personalized feedback.

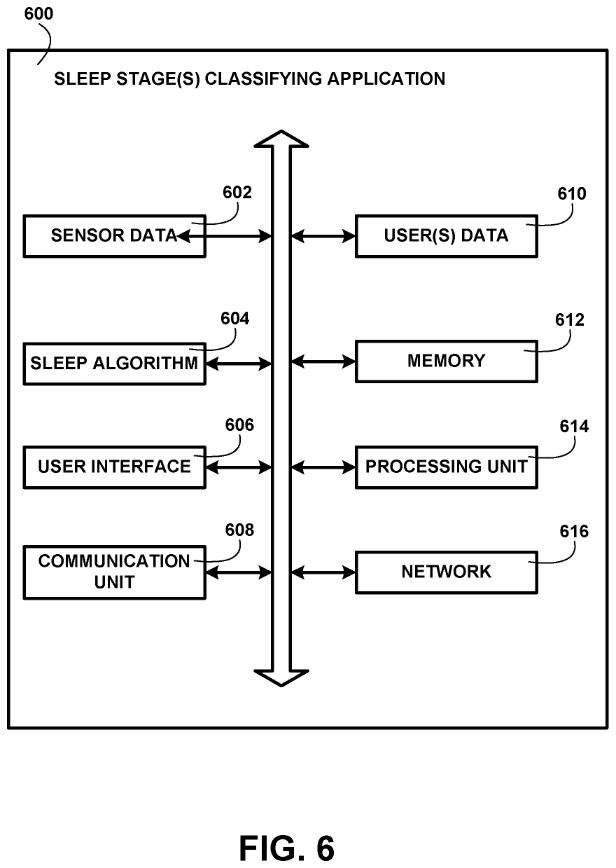

FIG. 6 shows, for illustrative purposes only, an example of a sleep analysis application, deployable with components distributed across one or more connected systems described in this disclosure, such as a wearable device, gateway device, and/or server(s) of one embodiment. FIG. 6 illustrates a block diagram of a sleep stage(s) classifying application 600 configured to receive sensor data 602 and process the sensor data 602 to determine sleep stage classifications. The sleep stage(s) classifying application 600 communicates with a user interface 604, a communication unit 606, user(s) data 608, memory 610, a processing unit 612, and a network 614. The processing unit 612 is configured to execute sleep staging algorithms using the sensor data 602 and user(s) data 608, and memory 610 is configured to store physiological data, extracted features, sleep stage classifications, and algorithm parameters. The communication unit 606 transmits and receives data between the sleep stage(s) classifying application 600 and external devices through the network 614, and the user interface 604 presents sleep stage information and related data to a user. The sleep stage(s) classifying application 600 integrates data from sensor data 602, user(s) data 608, and memory 610 to generate sleep stage classifications.

In one embodiment, the application, or portions thereof, may be deployed on a user's device 105 or on server(s) 150. Sensor data 602 are gathered from all connected devices. Depending on the type of sleep application and the demands of the model, the sleep stage(s) classifying application 600 may communicate with wearable device sensors, other sensor-based or sensor-less devices, or remote or local databases through the communication unit 606 and the network 614 to query data and retrieve the necessary information. user(s) data 608 may be provided by the user through the user interface 604, by filling out a questionnaire, or via any natural language prompting system.

In this context, the terms “sleep algorithm” and/or “sleep models” are used interchangeably, referring to a set of instructions or computational procedures applied to the received user(s) data 608 and sensor data 602 in the form of mathematical algorithms or computational instructions. When executed by the processing unit 612, these instructions result in the classification of a sequence of sleep stages. The classification process identifies at least one sleep class across the entire execution process, but multiple classifications, along with associated sleep scores and sleep stage confidence levels, may also be output from the algorithm.

The implementation may include local or remote memory 610 for storing the results of the classification or for invoking historical data collected from one or more users. The user interface 604 may come in various forms and designs, used to collect user(s) data 608, input information, and report results. The user interface 604 may include graphical diagrams, charts, plots, tables, figures, and means for downloading and syncing data to server(s) 150 or other connected devices through the communication unit 606 and the network 614. The user interface 604 may display various reports and specifications of the models, detailing their properties, confidence levels, and providing information on restfulness and sleep quality.

Processing unit 612, which is responsible for executing the algorithm, may be localized on the user's device 105 or distributed across processing units on server(s) 150 or cloud computing facilities. In some implementations, part of the processing activity may be carried out on the wearable devices themselves. The communication unit 606 enables the sleep stage(s) classifying application 600 to send and receive data, connect to other devices, and control those devices. The NETWORK 614 facilitates the system's connection to local networks, the internet, other gateway devices, other user devices, medical devices, internet server(s) 150, or local servers. In some implementations, the communication unit 606 enables connectivity through Bluetooth Low Energy (BLE), Wi-Fi, infrared, wired systems, radar, or other communication technologies.

In certain embodiments, the system comprises an end-to-end processing pipeline including sensor data acquisition, preprocessing, feature extraction, sleep algorithm execution, user interface generation, and communication with external systems. Sensor data acquired from one or more devices may be transmitted to a processing unit, where it is subjected to preprocessing and feature extraction operations. The processed data may then be provided as input to one or more sleep stage classification algorithms.

In certain embodiments, the sleep stage classification algorithms may operate locally on a user device, remotely on a server system, or in a distributed manner across multiple processing units. The resulting sleep stage outputs may be transmitted to a user interface device for presentation to the user. In certain embodiments, the system may also transmit data to remote systems for storage, analysis, or model training.

In certain embodiments, the pipeline may operate continuously or periodically, enabling real-time or near real-time monitoring of sleep stages. The system may further support feedback loops, wherein outputs of the sleep stage classification algorithms are used to adjust processing parameters, model weights, or data acquisition settings.

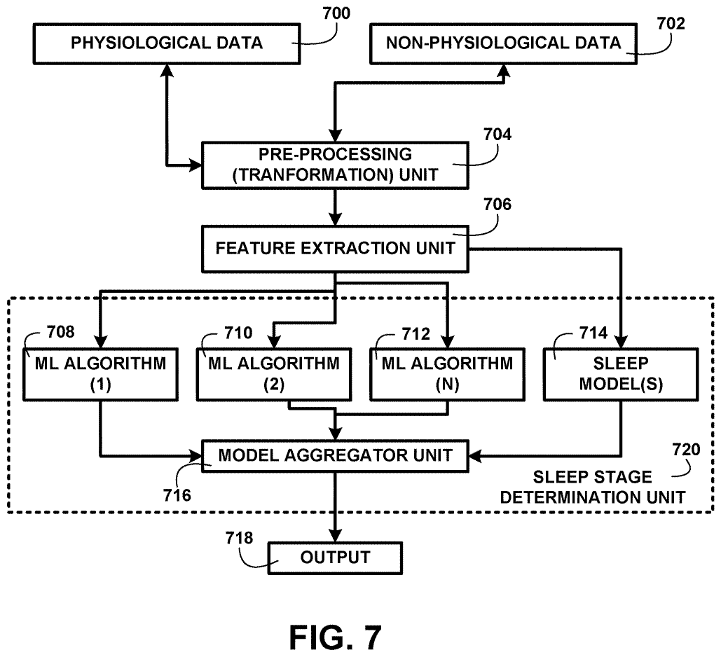

FIG. 7 shows, for illustrative purposes only, an example of a sleep staging system pipeline of one embodiment. FIG. 7 illustrates a sleep staging system pipeline in which physiological data 700 and non-physiological data 702 are provided to a pre-processing (transformation) unit 704. The pre-processing (transformation) unit 704 processes the physiological data 700 and non-physiological data 702 and provides processed data to a feature extraction unit 706. The feature extraction unit 706 extracts features from the processed data and provides extracted features to one or more sleep models, including ml algorithm (1) 710, ml algorithm (2) 712, and ml algorithm (n) 714, which together form sleep model(s) 708. Outputs from the sleep model(s) 708 are transmitted to a model aggregator unit 716, and the model aggregator unit 716 provides an output 718 to a sleep stage determination unit 720 for the determination of a sleep stage classification.

FIG. 7 illustrates an example of a sleep staging system pipeline. The pipeline begins with data acquisition from one or more physiological sensors, which collect physiological signals from the user over a period of time. These physiological signals may include, but are not limited to, motion data, temperature data, heart rate data, heart rate variability data, photoplethysmography signals, electrodermal activity signals, respiration data, and blood oxygen saturation data. The collected signals may be transmitted from a wearable device to a user device or a remote server for further processing.

Once the physiological signals are collected, the system performs signal preprocessing. Signal preprocessing may include filtering, noise reduction, signal normalization, resampling, interpolation, segmentation into sleep epochs, and artifact removal. The preprocessing stage prepares the physiological signals for feature extraction by improving signal quality and ensuring consistency across different sensor modalities.

Following preprocessing, the system performs feature extraction. Feature extraction involves computing relevant features from the physiological signals that may be used to classify sleep stages. These features may include time-domain features, frequency-domain features, statistical features, variability measures, signal amplitude features, signal slope features, entropy-based features, and other derived physiological metrics. The extracted features may be computed over fixed or dynamic time intervals corresponding to sleep epochs.

After feature extraction, the system performs feature selection and feature transformation. Feature selection may involve selecting a subset of relevant features based on feature importance scores, correlation analysis, or dimensionality reduction techniques. Feature transformation may include normalization, standardization, principal component analysis, clustering, or other mathematical transformations to improve model performance and reduce computational complexity.

The processed features are then provided to one or more sleep stage classification models. Each model may receive a different subset of features and may be configured to classify one or more sleep stages. The classification models may include machine learning models, deep learning models, probabilistic models, rule-based models, or mathematical models representing sleep behavior.

The outputs from the sleep stage classification models may include sleep stage predictions, probability scores, confidence scores, and other classification metrics. These outputs may then be processed by a post-processing unit that refines the classification results. Post-processing may include smoothing of sleep stage transitions, enforcement of transition rules, probabilistic modeling of sleep stage transitions, and correction of biologically implausible transitions.

The refined outputs are then provided to an output generation stage, where the final sleep stage classification results are generated. The output generation stage may also compute sleep metrics such as total sleep time, time spent in each sleep stage, sleep efficiency, sleep latency, wake after sleep onset, sleep score, readiness score, and other sleep-related metrics.

The final results may then be transmitted to a user interface for display to the user. The user interface may display graphical representations of sleep stages over time, numerical summaries, sleep scores, and recommendations for improving sleep quality. The results may also be stored in local or remote databases for long-term tracking, historical analysis, and model training.

In some implementations, the pipeline may operate in real time, where sleep stage predictions are generated continuously as new physiological data is received. In other implementations, the pipeline may operate retrospectively, where sleep stage classification is performed after the completion of a sleep session using stored physiological data.

The pipeline may also incorporate feedback mechanisms, where the results of the classification are used to update model parameters, improve feature selection, or adjust classification thresholds. This adaptive learning process allows the system to improve classification accuracy over time and adapt to individual user sleep patterns.

In certain embodiments, the system further comprises a preprocessing unit configured to receive raw physiological and non-physiological signals and to perform one or more signal conditioning operations prior to feature extraction. Such preprocessing operations may include filtering, normalization, artifact removal, segmentation, and resampling. For example, raw PPG signals may be processed using bandpass filtering to remove noise and motion artifacts, while accelerometer signals may be processed to isolate relevant motion components. In certain embodiments, preprocessing may also include detection and removal of outliers, interpolation of missing data, and alignment of signals collected from multiple sensors.

In certain embodiments, the system further comprises a feature extraction unit configured to generate one or more feature sets from the preprocessed signals. Such features may include time-domain features, frequency-domain features, and statistical features. For example, heart rate variability (HRV) features may include metrics such as root mean square of successive differences (rMSSD), standard deviation of NN intervals (SDNN), and frequency band power components. Motion features may include activity counts, movement intensity, and sleep-wake indicators derived from accelerometer data. Temperature features may include absolute skin temperature, rate of change, and circadian variation patterns.

In certain embodiments, the feature extraction unit may further perform transformation operations, including windowing, scaling, normalization, dimensionality reduction, or feature selection. For example, signals may be segmented into fixed-duration epochs, and features may be computed for each epoch to form input vectors for one or more machine learning models. In certain embodiments, the feature extraction unit may generate distinct feature sets for different models, enabling specialized models to operate on tailored subsets of input data.

In certain embodiments, the preprocessing unit and feature extraction unit operate in conjunction to transform raw sensor data into structured input representations suitable for machine learning-based sleep stage classification. These units may be implemented as software modules executed by a processing unit and may operate in real-time or batch processing modes.

In certain embodiments, the system further comprises a sensor fusion unit configured to receive, correlate, and combine data obtained from a plurality of heterogeneous sensor sources into a unified and temporally consistent representation suitable for sleep stage determination. The sensor fusion unit may be implemented as one or more software modules executed by a processing unit and stored in memory, and may operate on raw signals, partially processed signals, or feature-level representations derived from such signals.

In certain embodiments, the sensor fusion unit is configured to receive input data streams from multiple physiological and non-physiological sensors, including but not limited to photoplethysmography (PPG) signals indicative of blood volume changes, electrodermal activity (EDA) signals indicative of sympathetic nervous system activity, accelerometer-derived motion data indicative of body movement and posture, skin temperature signals indicative of peripheral thermoregulation, and environmental sensor data including ambient temperature, light exposure, or sound levels. The fusion unit may further receive contextual or user-generated data, including event markers or behavioral inputs.

In certain embodiments, the sensor fusion unit performs temporal alignment of signals obtained from different sensors, including resampling or interpolating signals to a common time base, correcting for clock drift, and synchronizing data streams across devices. In certain embodiments, amplitude normalization, baseline correction, and scaling operations may be applied to ensure that signals from different modalities are comparable and suitable for combined analysis.

In certain embodiments, the sensor fusion unit performs weighted fusion of input data, wherein weights are dynamically assigned to each signal or feature based on signal quality metrics, sensor reliability, contextual relevance, and historical performance. For example, during periods of low movement, motion-derived features may be assigned lower weight, while heart rate variability features derived from PPG signals may be assigned higher weight. Conversely, during periods of motion artifact contamination, motion data may be used to discount unreliable PPG-derived features.

In certain embodiments, fusion operations may be performed at multiple levels, including raw signal fusion, feature-level fusion, and decision-level fusion. Raw signal fusion may involve combining signals prior to feature extraction, while feature-level fusion may involve concatenating or transforming feature vectors derived from different sensors. Decision-level fusion may involve combining outputs of multiple models or classifiers, as described elsewhere herein.

In certain embodiments, the sensor fusion unit is further configured to detect, compensate for, and mitigate the effects of missing, degraded, or corrupted sensor data. For example, where a PPG signal is degraded due to motion artifacts or poor sensor contact, the system may rely more heavily on EDA, temperature, or motion-derived features. In certain embodiments, imputation techniques, predictive modeling, or redundancy across sensors may be used to reconstruct missing information. In this manner, the sensor fusion unit provides a robust and adaptive mechanism for integrating multi-modal data under varying conditions.

In certain embodiments, the system further comprises a signal quality assessment unit configured to evaluate the integrity, reliability, and usability of incoming sensor data prior to and/or during processing. The signal quality assessment unit may operate continuously or periodically and may be implemented as a software module executed by a processing unit.

In certain embodiments, the signal quality assessment unit computes one or more quality metrics for each signal, including but not limited to signal-to-noise ratio, motion artifact levels, signal amplitude stability, baseline drift, signal continuity, and completeness of data over a given time interval. In certain embodiments, quality metrics may also include sensor-specific indicators, such as optical signal strength for PPG sensors or electrode contact impedance for EDA sensors.

In certain embodiments, the system utilizes signal quality metrics to dynamically adjust processing operations. For example, signals determined to be below a predefined quality threshold may be excluded from feature extraction, down-weighted during sensor fusion, or replaced with alternative data sources. In certain embodiments, the system may selectively enable or disable certain models based on the availability and quality of required input signals.

In certain embodiments, the signal quality assessment unit may further identify transient artifacts, including motion-induced distortions, environmental interference, or sensor displacement events, and may apply corrective actions such as filtering, segmentation, or artifact removal. In certain embodiments, quality assessment may also trigger user notifications or device adjustments, such as prompting improved sensor placement.

In certain embodiments, signal quality metrics may be stored in memory and associated with corresponding data segments, enabling retrospective analysis, calibration of sensor systems, and refinement of model parameters. In this manner, the signal quality assessment unit enhances the robustness and reliability of the overall system.

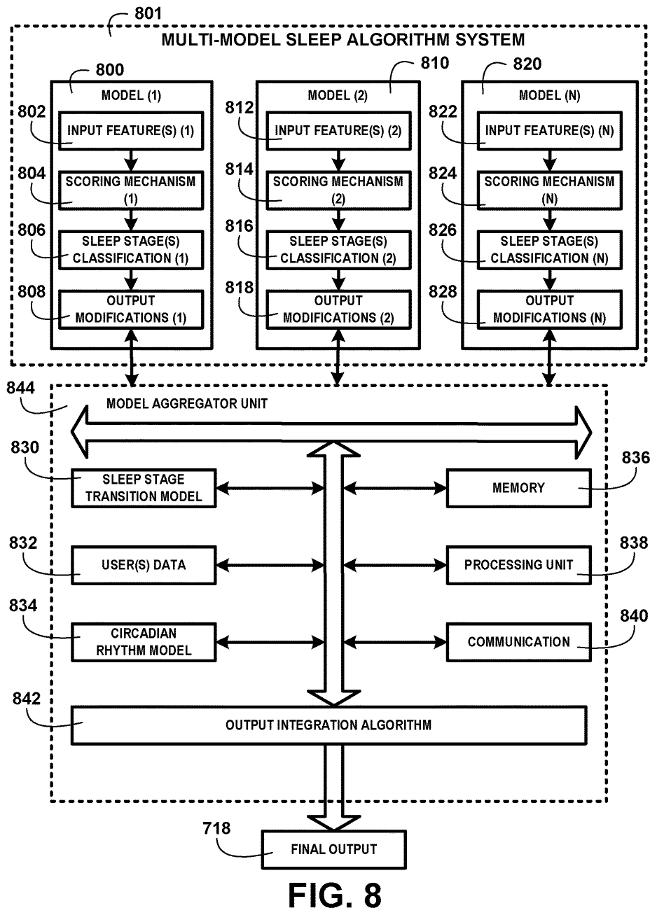

FIG. 8 shows, for illustrative purposes only, an example of a multi-model sleep algorithm, outlining examples of internal model structures and an output aggregator of one embodiment. FIG. 8 illustrates a multi-model sleep algorithm system 801 that includes a plurality of models, including model (1) 800, model (2) 810, and model (n) 820. Model (1) 800 receives input feature(s) (1) 802, which are processed by scoring mechanism (1) 804 to generate sleep stage(s) classification (1) 806, and the sleep stage(s) classification (1) 806 is processed by output modifications (1) 808. Model (2) 810 receives input feature(s) (2) 812, which are processed by scoring mechanism (2) 814 to generate sleep stage(s) classification (2) 816, and the sleep stage(s) classification (2) 816 is processed by output modifications (2) 818. Model (n) 820 receives input feature(s) (n) 822, which are processed by scoring mechanism (n) 824 to generate sleep stage(s) classification (n) 826, and the sleep stage(s) classification (n) 826 is processed by output modifications (n) 828.

Outputs from output modifications (1) 808, output modifications (2) 818, and output modifications (n) 828 are transmitted to a model aggregator unit 844, and the model aggregator unit 844 provides aggregated outputs to an output integration algorithm 842 to generate a final output 718.

FIG. 8 illustrates an example implementation detailing the sleep staging models (720, 730 of FIG. 7). The model pipeline receives specific input features 802 tailored to each model, ensuring that relevant physiological and contextual data are processed appropriately. Based on the quality of the data used to generate the feature set, a scoring mechanism 804 calculates the importance of each model and the significance or confidence level of its results when aggregating multiple model outputs within the model aggregator mechanism. This scoring mechanism 804 ensures that higher-weighted models contribute more prominently to the final sleep classification, while models with lower confidence scores are either down-weighted or excluded from the aggregation process.

The sleep stage classifiers 806 may be implemented as either machine learning-based models trained to generate sleep stage predictions based on the input features or as non-machine learning models that mathematically represent sleep patterns. These classifiers may incorporate circadian rhythm data, user-reported experiences, recent activity levels, and other health-related factors to refine their predictions. For example, a user who recently experienced jet lag may have altered sleep patterns that influence the classification process. In such cases, a circadian model may adjust sleep stage transitions to account for potential disruptions caused by travel.

The output modification unit 810 is responsible for post-processing the sleep stage sequences generated by the classifiers. Various post-processing models, such as Hidden Markov Models (HMMs), may be utilized to smooth transitions between sleep stages, filter out noisy predictions, and assign confidence levels to each classified sleep stage. By incorporating probabilistic models such as HMMs, the system ensures that abrupt or biologically implausible sleep stage transitions are corrected, resulting in a more realistic and clinically relevant sleep profile. Additionally, post-processing mechanisms may incorporate temporal constraints to ensure consistency across sleep cycles.

In some implementations, sleep models 720 may not only operate independently but also engage in iterative collaboration, enabling them to refine predictions through mutual learning and feature exchange. These models may leverage latent representations of physiological signals, generate supplementary features for one another, or influence each other's scoring mechanisms to enhance classification accuracy. The iterative execution of sleep models allows for progressive refinement of sleep stage predictions, with each iteration incorporating additional contextual and historical data to improve overall performance. For example, an initial classification pass may generate a preliminary sleep stage sequence, which is subsequently refined by integrating additional physiological trends, user-reported inputs, or circadian rhythm data. This dynamic approach allows the system to adapt to user-specific variations in sleep patterns, ultimately leading to a more personalized and precise assessment of sleep architecture.

The model aggregator unit 740, as illustrated in further detail in FIG. 8, is responsible for consolidating outputs from multiple sleep models to produce a final sleep stage classification. This aggregation process takes into account model-specific scores generated by the scoring mechanism 804, ensuring that models with higher confidence and accuracy contribute more significantly to the final classification. In some implementations, the model aggregator unit 740 may access additional historical data from the same user or other users within the system to enhance classification robustness. This data acquisition may be facilitated by a communication unit 840, which enables real-time retrieval of relevant sleep records, physiological parameters, and contextual information stored in remote or local databases.

In certain implementations, the model aggregator unit 740 may also incorporate a circadian rhythm model 834, which operates as an independent analytical component. The circadian rhythm model provides insights into expected sleep-wake patterns based on biological rhythms, external environmental cues, and user-reported behavioral patterns. By integrating circadian information, the system can further refine sleep stage classifications, particularly in cases where conventional physiological markers alone may be insufficient for accurate classification. The model may analyze factors such as melatonin secretion cycles, light exposure, and habitual sleep schedules to adjust predicted sleep stages accordingly.

User data 825, either explicitly provided by the user or retrieved from health-related databases, may be utilized to further enhance the accuracy of sleep classification. Such data may include medical history, medication usage, stress levels, recent physical activity, and other health-related factors that influence sleep patterns. By incorporating these additional contextual variables, the model aggregator unit 740 provides a more holistic and individualized sleep analysis.

The output aggregator algorithm 842 processes all gathered information and produces the final sleep stage summary. During this phase, the system calculates a final confidence score, assessing the reliability of the classification results. Additionally, the algorithm performs reevaluation and sanity checks on all generated outputs, ensuring consistency, eliminating anomalies, and validating transitions between sleep stages.

This final validation step helps mitigate errors that may arise due to sensor noise, inconsistent model predictions, or unexpected physiological fluctuations.

The sleep transition model, which may be implemented using a Hidden Markov Model (HMM) or similar probabilistic frameworks, serves to refine the classification of sleep stages by capturing temporal dependencies in sleep patterns. HMMs, in particular, are well-suited for modeling sequential data, such as sleep stage transitions, due to their ability to capture temporal dependencies and probabilistic relationships between states. The model utilizes posterior and prior probabilities to determine the likelihood of a given sleep stage transition occurring.

In this approach, the model operates by estimating the probability of transitioning from one sleep stage to another based on prior knowledge of typical sleep patterns, either derived for the same user or an ensemble of users, and real-time sensor observations. The prior probability represents the likelihood of a particular sleep stage occurring before considering new sensor data, drawing from historical sleep data, established sleep cycle structures, and individual sleep habits. The posterior probability, in contrast, is updated as new physiological signals are received, incorporating real-time measurements to refine the classification of sleep stages in a way that reflects both the expected sleep architecture and the user's actual physiological responses.

For instance, if a user is detected in deep sleep during one time interval, the probability of transitioning directly to wakefulness in the next interval would typically be lower than transitioning to lighter sleep stages, based on known sleep progression patterns. The model considers these transition probabilities when making classification decisions, favoring sequences that align with natural sleep progression rather than abrupt or physiologically unlikely shifts.

To further improve classification accuracy, the sleep transition model may incorporate a smoothing mechanism that adjusts classifications by evaluating both past and future stages in a given sleep period. This retrospective and forward-looking analysis helps to refine stage assignments by reducing the influence of brief anomalies, such as a sudden spike in heart rate incorrectly suggesting wakefulness when the broader context indicates continued sleep.

By integrating prior expectations of sleep stage transitions with real-time physiological data, the model adapts to individual variations while maintaining a logical flow in sleep stage classification. The ability to incorporate both general sleep science principles and personalized sleep dynamics allows the system to produce a more stable and clinically meaningful representation of sleep structure.

Additionally, sleep transition models may employ Viterbi decoding to determine the most probable sequence of sleep stages over a given sleep period. By analyzing the sequence of observations and maximizing the joint probability of sleep stage transitions, the system can enhance the temporal coherence of the classified sleep stages. This approach ensures that sleep stage sequences adhere to physiological norms and reduces classification errors arising from momentary fluctuations in sensor readings.

Overall, by integrating machine learning models, circadian rhythm models, user-provided contextual data, and probabilistic sleep transition models, the system ensures a highly accurate and personalized approach to sleep stage classification. The iterative and collaborative nature of the models, coupled with robust aggregation and post-processing mechanisms, enables the generation of reliable, clinically relevant sleep assessments that can be used for further sleep health analysis and optimization.

Once the sleep stage(s) classifier 600 has processed the physiological data, the system proceeds to classify the data into one or more sleep stages that may include but are not limited to awake, light sleep, REM sleep, and deep sleep. This classification is derived from at least one physiological signal and/or other data collected throughout the user's sleep period, enabling the system to determine and assign specific sleep stages based on the detected patterns. At least one sleep stage, such as light sleep, REM sleep, or deep sleep, is identified within the classification process, with the potential for further granularity depending on the model's capabilities and the quality of the input data.

Following classification, the user personal device 105 may present the classified sleep intervals to the user via a graphical interface, detailing the duration of each sleep stage and potentially offering additional insights into sleep quality, restfulness, and sleep cycle progression. These outputs may be supplemented with model-derived interpretations, classifications, and extracted latent features, which could include hidden states of the model, transition probabilities between sleep stages, confidence levels, and other relevant sleep metrics. Such information may be reported to the user in real-time as data is processed or retrospectively at the conclusion of the sleep session, providing a comprehensive summary of sleep patterns and trends over time.

In some cases, the user personal device 105 may also display additional information, such as the user's average heart rate, HRV, temperature, and other relevant metrics. These metrics provide users with a holistic view of their sleep patterns and overall health, helping them make informed decisions about their sleep habits.

The system 600 can be also designed to train machine learning classifiers using physiological data collected from each individual user. This enables the creation of machine learning models that are tailored to each user's specific characteristics. For example, as mentioned earlier, the system 300 can collect data from a user during the first night of sleep (Night 1) and classify it into sleep stages using a machine learning classifier. On the following night (Night 2), the system collects additional physiological data, which is input into the machine learning classifier to classify the new data. The classifier can then combine the data from Night 1 and Night 2 to classify the sleep stages, and this process can be repeated for multiple nights. This continuous training process helps to incrementally improve the classifier's accuracy over time, making it more reliable in identifying sleep stages as more data is gathered from the user.

The machine learning classifier can utilize a variety of parameters and features from the collected physiological data to classify sleep stages. For example, the classifier may rely solely on accelerometer data (ACC model) in some cases. In other scenarios, it may use both accelerometer and temperature data (ACC+T model) or incorporate accelerometer, temperature, and heart rate variability (HRV) data (ACC+T+HRV model). Additional physiological parameters, such as blood oxygen levels (SpO2), pulse waveforms, respiration rate, pulse oximetry, and blood pressure, may also be used in the classification process.

In the case of two-stage classification (e.g., classifying sleep into awake and asleep stages), accelerometer-based models (ACC model) achieved 94% accuracy with an F1 score of 0.67. Including temperature data (ACC+T model) resulted in a slight improvement to 95% accuracy (F1score=0.69). When HRV data was added (ACC+T+HRV model), accuracy increased to 96% (F1score=0.76). Including circadian features further boosted accuracy to 96% with an F1 score of 0.78. For four-stage classification (e.g., awake, light, REM, and deep sleep), accelerometer-based models (ACC model) exhibited 57% accuracy (F1score=0.68). Including temperature (ACC+T model) led to a slight increase in accuracy to 60% (F1score=0.69). Adding HRV data (ACC+T+HRV model) improved accuracy to 76% (F1score=0.73), and adding circadian features (ACC+T+HRV+C model) raised accuracy to 78% (F1score=0.78).

In some implementations, the system 300 may further leverage circadian features in the classification process. Circadian rhythm modeling accounts for variations in sleep stage frequency across the night, as the circadian rhythm influences sleep patterns. The circadian rhythm refers to a natural, internal process that regulates the sleep-wake cycle, typically repeating every 24 hours. For example, during the night, deep sleep tends to occur more frequently at the beginning, while REM sleep is more prevalent in the latter portion of the night. By incorporating the time elapsed during the night, the time of day, and the individual's circadian rhythm into the classification process, the system can better account for these patterns and improve classification accuracy. For example, when circadian features are included, the system achieved 96% accuracy in two-stage classification (F1score=0.78) and 78% accuracy in four-stage classification (F1score=0.78).

In certain implementations, the sleep stage classification application 600 may be executed entirely on server(s) 150, where the classification process is performed locally or remotely, and the results are subsequently transmitted to and displayed on the user's device 105. This approach allows for computationally intensive sleep classification models to be deployed without burdening the user's device 105 with excessive processing demands. Additionally, the execution of sleep stage classification may occur retrospectively, whereby an updated version of the sleep models and algorithms is applied to historical sleep data stored in system memory or databases. This retrospective classification may be used to refine previously determined sleep stages, recalibrate sleep metrics using enhanced models, or generate new insights from past sleep sessions by leveraging advancements in machine learning algorithms or novel sleep analysis techniques.

In some variations of the system, the machine learning algorithms 720 and the sleep models 730 may each classify a subset of sleep stages independently, after which the aggregation process may be applied to integrate the results into a broader classification covering multiple sleep stages. In such an implementation, different models may specialize in specific sleep characteristics, with some models focusing on distinguishing between major sleep stages such as REM sleep, deep sleep, and light sleep, while others analyze finer details within sleep physiology, leading to novel and refined sleep stage categorizations. These alternative or newly derived sleep stages may emerge from patterns identified in physiological data that are not explicitly defined by traditional sleep science but are nonetheless indicative of sleep health, recovery, or other relevant aspects of the user's sleep performance. These latent or novel sleep stages may be directly or indirectly associated with conventional sleep stages, such as REM sleep, non-REM sleep, deep sleep, light sleep, and wakefulness, but may also introduce additional classifications that provide further granularity and insight into the user's sleep architecture.

In some implementations, machine learning algorithms 720 may incorporate bidirectional modeling techniques, wherein the classification process is not limited to past sleep stage transitions but also accounts for future sleep stages occurring within the sleep session, sleep cycle, or sleep epoch. In such cases, the model does not function strictly as a causal system that derives sleep stages solely from the currently collected real-time signals (or data) and the previously determined sleep stage patterns. Instead, it may employ bidirectional inference mechanisms to improve classification accuracy by integrating both preceding and forthcoming sleep-related information. The aggregation unit and sleep transition models may further enhance this bidirectional framework by refining the inference of sleep stage sequences, utilizing both retrospective and prospective data to generate a more comprehensive and accurate classification.

An exemplary approach to such an implementation may include the use of bidirectional recurrent neural networks, such as bidirectional long short-term memory (BiLSTM) networks, which process sleep data by capturing temporal dependencies in both forward and backward directions. Unlike unidirectional models that can only learn from past time steps, bidirectional models allow for a more comprehensive analysis by incorporating information from both preceding and subsequent sleep periods. Additionally, in some versions, attention mechanisms may be integrated with bidirectional models to assign varying levels of importance to different segments of the sleep period, enabling the system to focus on regions of the sleep session that contain more critical information regarding sleep transitions, anomalies, or significant physiological changes. This approach enhances the model's ability to detect subtle sleep patterns that may be less apparent in traditional unidirectional classification models.

In some implementations, the system 300 can input a circadian rhythm adjustment model into the machine learning classifier. This model helps the classifier classify physiological data into corresponding sleep stages based on the user's circadian rhythm. The circadian rhythm adjustment model can “weight” the physiological data depending on the user's circadian rhythm. This means that the model can influence how likely certain time intervals or physiological data are to correspond to a particular sleep stage, considering the natural variation in sleep stages over the course of the night.

For instance, because deep sleep typically occurs more often during the first part of the night and REM sleep is more frequent toward the end, the circadian rhythm adjustment model 835 can adjust the probability of time intervals early in the night being classified as deep sleep and time intervals later in the night being classified as REM sleep. This can be done by accounting for physiological markers such as heart rate and breathing rate variability, which are associated with different sleep stages. In practice, this means that the algorithm may assign a higher probability of deep sleep to earlier time periods when the user is likely experiencing lower heart rate and more consistent breathing patterns, whereas the model may adjust for higher likelihoods of REM sleep later in the night when these physiological patterns change.

Furthermore, the circadian rhythm adjustment model may be personalized for each user. Physiological data collected from the user can help refine a baseline circadian rhythm adjustment model, tailoring it to that user's specific sleep patterns. This personalized model could adjust to account for the individual's sleep preferences and behaviors. For example, if the system detects a user's habitual bedtime or sleep duration, it can modify the circadian rhythm adjustment model accordingly.

The system may also utilize generalized circadian rhythm adjustment models that are based on data from multiple users, providing a starting point for sleep stage classification. However, these generalized models can be further customized to each user by incorporating their unique physiological data. By continuously refining the model with new data, the system can improve its predictions and better reflect each user's sleep habits and needs.

The circadian rhythm adjustment model may include several components to capture both the circadian rhythm and the homeostatic sleep drive. These components include a circadian drive component, a homeostatic sleep pressure component, and an elapsed sleep duration component. The circadian drive component models the natural cycle of sleep and wakefulness, which follows a roughly 24-hour rhythm. The homeostatic sleep pressure component represents the increasing need for sleep the longer a person stays awake, which is counteracted by the restorative effects of sleep. The elapsed sleep duration component models how the progression of time influences sleep stage distribution throughout the night, taking into account the fact that sleep patterns are not uniform and that different sleep stages are more likely to occur at different times.

The circadian rhythm adjustment model helps to account for real-world variability in sleep schedules, such as differences in weekday and weekend sleep times, travel, or shift work. The system adjusts for these factors by tracking each user's sleep behavior and continuously modifying the circadian rhythm model to reflect changes in their sleep patterns.

The combination of multiple physiological data streams, circadian rhythm features, and machine learning techniques allows the system to classify sleep stages with high accuracy. The system's performance, especially when using accelerometer, temperature, HRV, and circadian features, has been shown to approach the accuracy of EEG-based systems in detecting sleep stages. By integrating data from wearable sensors, like the watch 101, and using advanced machine learning algorithms, the system achieves high sensitivity and specificity in detecting various sleep stages and wakefulness, which improves the overall reliability of sleep tracking.

The use of accelerometer-only data (ACC models) has significantly improved the accuracy of typical sleep and wake detection systems, which traditionally rely on actigraphy and basic motion-intensity features. Accelerometer models have been shown to offer better performance by utilizing more advanced physiological data, such as temperature, heart rate, and heart rate variability (HRV). These additional parameters provide a finer discrimination between different sleep stages and are less prone to issues like calibration errors or hardware discrepancies. For instance, accelerometer data can capture relative deviations from previous windows or leverage trigonometric identities to estimate finger-derived motion in a more robust manner. This approach is less affected by potential disturbances, such as movement from a person's partner or pet in bed, making it a more reliable method for detecting sleep stages, especially when compared to simpler motion detection techniques.

While accelerometer-only models are still not on par with gold-standard PSG (polysomnography) in terms of accuracy-especially for four-stage classification-using the described advanced features has led to notable improvements. These enhanced models have shown better performance in detecting and classifying sleep stages, including deep NREM (Non-Rapid Eye Movement) sleep, which has traditionally been a challenge for consumer devices. Not only are wake states more accurately detected, but deeper sleep stages are now being classified with higher accuracy.

In addition to accelerometer data, incorporating peripheral finger temperature measurements has proven valuable in improving sleep staging accuracy. As previously noted, there is an inverse pattern with core body temperature, where temperature increases during the night and decreases during the day. Sleep onset is more likely to occur when core body temperature is at its steepest rate of decline. After sleep onset is determined, adding peripheral temperature measurements-such as those collected by wearable devices like the wearable device 101-can further enhance the accuracy of sleep stage classification. This data feature, often overlooked, remains an important signal for determining sleep onset and offset.

The most significant improvements in sleep stage classification accuracy are observed when HRV features are included. The wearable watch 101 uses optical technology to capture beat-to-beat intervals, allowing for the calculation of heart rate and more advanced HRV features, which can be used to estimate sleep stages. HRV features offer a tighter connection to the central nervous system's activity and changes in the autonomic nervous system (ANS), which can be captured non-invasively. These physiological changes are consistent with specific sleep stages, particularly in distinguishing between NREM and REM (Rapid Eye Movement) sleep. During REM sleep, heart rate tends to increase and exhibit greater variability, while during NREM sleep, both heart rate and HRV generally decrease. Including heart rate data can improve four-stage classification accuracy by 15-25%, and the addition of HRV features indicative of parasympathetic activity during NREM sleep can further enhance performance. These HRV patterns reflect underlying changes in brain activity, similar to those captured by EEG-based PSG systems, which makes HRV an essential parameter for improving classification accuracy.

The distribution of sleep stages across the night exhibits both expected and idiosyncratic patterns. Typically, sleep cycles last between 70 and 120 minutes, with deep NREM sleep being more prevalent in the first third of the night and REM sleep more concentrated in the second half. Additionally, each REM bout becomes progressively longer as the night continues. Accounting for the waxing-and-waning of the circadian rhythm throughout the night, alongside the decay of homeostatic sleep pressure and the elapsed time since sleep onset, can significantly improve classification accuracy-up to 78% in some cases. Previous sleep stage detection methods have attempted to model temporal associations between stages using techniques like Markov models and neural networks. However, incorporating circadian features, which are sensor-independent, provides a more effective means of accounting for these temporal variations, leading to better performance.

The system also integrates a range of advanced components in wearable applications to support these sleep staging algorithms. As illustrated in a block diagram of the device, a mobile or wearable device may include an input module, output module, and wearable application components. These components work together to acquire physiological data, process the data, and display the sleep stages on a user interface. The input module manages signals received from the wearable device or other sources, while the output module manages the transmission of processed signals for storage or further analysis. The wearable application itself may include data acquisition components, machine learning classifier components, and user interface components to facilitate real-time data collection, classification, and feedback.

The wearable application's data acquisition component is responsible for gathering physiological data from the user through wearable devices, such as wristbands or rings. The machine learning classifier component then processes this data to classify it into one of several sleep stages, such as awake, light sleep, REM sleep, or deep sleep. The user interface component allows the device to display this classification on the user's device, providing valuable insights into their sleep patterns. In some cases, the system may also include additional components like data normalization, user evaluation, and data transmission to ensure smooth operation and provide further insights into the user's sleep behavior.

Through these various components, the wearable application enables users to track and analyze their sleep with high accuracy, offering a sophisticated method of detecting and classifying sleep stages. The integration of advanced features like HRV, temperature, and circadian rhythm adjustments, along with machine learning techniques, allows the system to rival traditional PSG systems in terms of classification performance, providing users with a comprehensive and reliable sleep monitoring experience.

In some embodiments, the data acquisition component 305 may be configured to receive additional physiological data associated with a user from the wearable watch device, with the physiological data being collected via the wearable watch device throughout a second time interval. This additional data may include a variety of physiological parameters, such as heart rate, blood oxygen levels, and other relevant biometrics. The data acquisition component 832 may be configured to manage and facilitate the transmission of this additional physiological data for further processing.

In some instances, the machine learning classifier component 720 may be configured to receive the additional physiological data as input. The machine learning classifier component 720 may be configured to classify the physiological data, including the additional data, into one or more sleep stages from a plurality of sleep stages over at least a portion of the second time interval. The classification of the physiological data into sleep stages may rely on a combination of the initial physiological data and the additional data collected during the second time interval. The classification process is at least partially performed by the machine learning classifier, utilizing its trained models to assess the user's sleep patterns and stages based on the received physiological data.

Furthermore, the user interface component 630 may be configured to control a graphical user interface (GUI) of a user device, such that it displays an indication of the classified sleep stages. The GUI may show one or more sleep stages for the second time interval, based at least in part on the classification of the additional physiological data. The user interface component 630 may be responsible for ensuring that the user is presented with clear and accurate visual feedback regarding their sleep stages, facilitating further analysis or adjustments by the user.

In some embodiments, the user interface component 630 may also support the display of at least a subset of the physiological data collected, such as temperature data, accelerometer data, heart rate data, heart rate variability data, or blood oxygen levels. In some instances, the wearable wristband device may collect this physiological data through the measurement of arterial blood flow within a user's finger. The device may use one or more red LEDs and one or more green LEDs to capture this data, providing a reliable and continuous measurement of key biometrics relevant for sleep stage classification.

Moreover, in certain examples, the system may apply a circadian rhythm adjustment model. This model accounts for the natural fluctuations in a user's sleep patterns based on their biological clock and may help adjust the classification of sleep stages by considering the time of night, historical sleep data, and other individual factors. Circadian rhythm models may use predefined patterns of sleep onset and wake times, as well as typical durations for each stage, to help predict the most probable state transitions.

The system may also incorporate advanced features such as normalization of physiological data. The normalization process may ensure that variations in the sensor data, such as differing sensitivity or noise across different devices, do not affect the accuracy of the sleep stage classification. Normalization procedures may involve scaling the data to a uniform range, removing outliers, or adjusting the data to account for individual baseline differences, such as heart rate variability between users.

Moreover, the machine learning classifier may identify key features associated with the physiological data, including the rate of change of physiological signals, patterns between multiple sensor outputs, or statistical properties of the data (e.g., maximum, minimum, median, or average values). These features are used to enhance the accuracy of the sleep stage classification, allowing for the identification of nuanced physiological changes that may signal transitions between different sleep stages.

In some embodiments, the method may also include the ability to generate actionable sleep scores based on the physiological data and sleep stage classification. These scores, such as a Sleep Score or Readiness Score, may be calculated using a weighted formula that takes into account time spent in each sleep stage, overall sleep duration, and sleep continuity. The GUI may then display these scores, providing users with an easy-to-understand summary of their sleep quality.

In other variations, the method may include an adaptive feedback loop, where the classifier refines its predictions based on user feedback or contextual data. This feedback loop may improve the long-term accuracy of the system, enabling it to better capture individual differences in sleep physiology.

The system may allow for the integration of multiple data sources, such as other wearables or environmental sensors, which can further improve the classification accuracy by providing additional context or data that may not be captured by the wearable wristband device 101 alone. This multi-sensor approach ensures that the classification process is more robust and provides a comprehensive view of the user's sleep patterns.

The sleep staging system 100 described in this disclosure may possess the capability to interface with medical-grade devices or access databases generated by such devices in order.

The embodiments described herein may be implemented using hardware, software, firmware, or combinations thereof, and may be implemented on one or more computing devices or distributed across multiple interconnected computing devices. The specific components, modules, devices, and processing steps described herein are provided for illustrative purposes, and other configurations, combinations of components, and processing sequences may be used without departing from the scope of the invention. The system may be adapted for different types of sensors, different types of input data, different processing algorithms, and different output requirements depending on the application.

It will be understood that the system may be configured to operate with various types of computing devices, sensors, communication systems, artificial intelligence systems, and machine learning systems, and that the arrangement of components described herein may be modified, combined, or distributed across multiple devices. Accordingly, the description is intended to be illustrative rather than limiting, and the scope of the invention is defined by the claims.

In certain embodiments, the system further comprises a model aggregator unit configured to receive, evaluate, and integrate outputs generated by a plurality of independently trained and/or concurrently operating sleep stage classification models. Each of the plurality of models may be configured to process one or more distinct sets of input features derived from physiological and/or non-physiological signals, including but not limited to photoplethysmography (PPG) signals, electrodermal activity (EDA) signals, accelerometer-derived motion data, skin temperature signals, heart rate data, heart rate variability (HRV) metrics, and contextual or event-based user inputs. The model aggregator unit may be implemented as a software module executed by one or more processing units, and may further be stored in memory and executed as a sequence of machine-readable instructions.

In certain embodiments, each model of the plurality of models generates a corresponding sleep stage classification output for a defined time interval or epoch, together with an associated confidence value, probability distribution, or likelihood score. The model aggregator unit is configured to receive these outputs and to perform one or more aggregation operations, including but not limited to weighted averaging, voting-based selection, probabilistic fusion, or rule-based combination. In some embodiments, the aggregation operation may assign weights to each model output based on factors including historical model accuracy, signal quality metrics, user-specific calibration parameters, or contextual conditions such as time of day or detected user activity.

In certain embodiments, the model aggregator unit is further configured to resolve conflicts between outputs of different models. For example, where a first model indicates a REM sleep stage and a second model indicates a non-REM stage for the same epoch, the model aggregator unit may apply conflict resolution logic based on confidence scores, transition constraints, historical patterns, or predefined prioritization rules. In some embodiments, the aggregator may discard or down-weight model outputs that are determined to be inconsistent with physiological plausibility constraints or temporal continuity constraints.

In certain embodiments, the model aggregator unit further performs validation and consistency checks on aggregated outputs. Such validation may include enforcing permissible sleep stage transitions, rejecting transient or spurious classifications caused by signal artifacts, and smoothing output sequences across consecutive epochs. In certain embodiments, the aggregated output may be refined using post-processing operations including temporal filtering, statistical smoothing, or constraint-based correction to generate a final sleep stage determination output.

In certain embodiments, the model aggregator unit is also configured to incorporate historical user data and/or population-level data in the aggregation process. For example, prior sleep patterns of a user, circadian rhythm models, or learned behavioral patterns may be used to influence weighting, filtering, or final classification decisions. In this manner, the model aggregator unit provides a structured mechanism for integrating outputs of multiple heterogeneous models into a unified and physiologically consistent sleep stage determination.

In certain embodiments, the system further comprises a sleep stage transition model configured to evaluate temporal relationships between consecutive sleep stage determinations and to enforce probabilistic constraints on allowable transitions. The sleep stage transition model may be implemented using a probabilistic graphical model, such as a Hidden Markov Model (HMM), or other temporal modeling techniques capable of representing sequential dependencies between sleep stages over time.

In certain embodiments, the sleep stage transition model defines a set of states corresponding to sleep stages, including but not limited to wake, REM, N1, N2, N3, and optionally additional stages or sub-stages. The model further defines transition probabilities between these states, representing the likelihood of transitioning from one stage to another over successive epochs. These transition probabilities may be predetermined based on known physiological sleep patterns, learned from training data, or dynamically adjusted based on user-specific historical data.

In certain embodiments, the system computes, for each epoch, a set of observation likelihoods derived from outputs of the plurality of sleep stage classification models. The sleep stage transition model is configured to combine these observation likelihoods with transition probabilities to determine a most probable sequence of sleep stages over time. In certain embodiments, this determination may be performed using algorithms such as the Viterbi algorithm, forward-backward inference, or other sequence decoding techniques.

In certain embodiments, the sleep stage transition model is further configured to enforce temporal smoothing and continuity constraints. For example, abrupt or physiologically implausible transitions between non-adjacent sleep stages may be suppressed or corrected. The model may also incorporate prior probabilities based on circadian rhythm phase, elapsed sleep duration, or user-specific sleep architecture patterns. In this manner, the sleep stage transition model operates in conjunction with the model aggregator unit to produce temporally consistent and physiologically plausible sleep stage outputs.

In certain embodiments, the plurality of sleep stage classification models are configured to operate in an iterative and/or collaborative manner. For example, outputs generated by one model may be used as input features or contextual information for another model. In certain embodiments, an initial set of model outputs may be refined through one or more subsequent processing passes, wherein later-stage models adjust or correct earlier classifications based on additional features, temporal context, or aggregated outputs.

In certain embodiments, the system may implement a multi-pass processing architecture in which intermediate outputs are iteratively updated until convergence criteria are satisfied or a predefined number of iterations is reached. During such iterative processing, models may exchange information, such as confidence scores, feature importance values, or intermediate classifications, thereby enabling cross-model refinement and improved overall accuracy.

In certain embodiments, collaborative behavior between models may include the sharing of feature representations, joint optimization of model parameters, or coordinated decision-making through the model aggregator unit. In this manner, the system is not limited to independent model operation, but instead supports coordinated and adaptive multi-model processing for sleep stage determination.

In certain embodiments, the system is configured to perform user-specific personalization of sleep stage classification by adapting one or more models, parameters, or processing operations based on historical data associated with an individual user. Such personalization may account for inter-user variability in physiological signals, sleep architecture, and behavioral patterns.

In certain embodiments, personalization may include adjusting model parameters, feature weights, classification thresholds, or aggregation rules based on observed patterns in a user's historical data. For example, variations in baseline heart rate variability, skin temperature ranges, or movement patterns may be incorporated into personalized models to improve classification accuracy.

In certain embodiments, personalization may be implemented using machine learning techniques, including supervised learning based on labeled user data, unsupervised clustering of user-specific patterns, or incremental learning approaches that update model parameters over time. In certain embodiments, the system may maintain one or more user-specific model instances or may apply personalization layers on top of generalized models trained on population data.

In certain embodiments, personalization may further incorporate user-provided inputs, including sleep diaries, subjective sleep quality ratings, event markers, or behavioral data. These inputs may be correlated with physiological signals to refine model outputs and improve predictive performance.

In certain embodiments, personalization may be continuously updated as new data is collected, enabling adaptive and evolving models that reflect changes in user behavior, health conditions, or environmental factors.

In certain embodiments, the system further comprises a circadian rhythm model configured to represent time-dependent variations in physiological processes that influence sleep-wake cycles. The circadian rhythm model may account for endogenous biological rhythms, including hormonal cycles, body temperature fluctuations, and sleep propensity patterns.

In certain embodiments, the system also incorporates a homeostatic sleep drive model representing the accumulation and dissipation of sleep pressure as a function of time awake and time asleep. The interaction between circadian and homeostatic processes may be used to influence sleep stage classification, transition probabilities, and confidence scoring.

In certain embodiments, circadian phase information may be derived from historical sleep patterns, environmental light exposure, user inputs, or external data sources. The system may use this information to bias classification outputs, enforce temporal constraints, or suppress physiologically implausible stage transitions.

In certain embodiments, circadian and homeostatic models may be integrated with the sleep stage transition model and model aggregator unit to provide a comprehensive and physiologically grounded framework for sleep stage determination.

In certain embodiments, the system supports the training, validation, and updating of machine learning models used for sleep stage classification. Training may be performed using datasets comprising physiological signals, annotated sleep stages, and contextual information obtained from clinical studies, user data, or aggregated population data.

In certain embodiments, training processes may include feature selection, model optimization, hyperparameter tuning, and validation using cross-validation or holdout datasets. The system may employ various machine learning techniques, including neural networks, decision trees, support vector machines, or probabilistic models.

In certain embodiments, model training may be performed on remote server systems, while inference is performed locally on user devices. In certain embodiments, models may be periodically updated based on newly collected data, enabling continuous improvement.

In certain embodiments, distributed or federated learning approaches may be used to update models across multiple devices without centralized collection of raw user data.

In certain embodiments, the system implements security mechanisms to protect user data during collection, transmission, storage, and processing. Such mechanisms may include encryption, authentication, and access control policies.

In certain embodiments, data may be encrypted using secure communication protocols during transmission and stored in encrypted form. Authentication mechanisms may ensure that only authorized devices and users can access system resources.

In certain embodiments, the system may anonymize or pseudonymize data prior to analysis or storage. Users may be provided with control over data usage, including options to enable or disable certain data collection features.

In certain embodiments, the system may operate in real-time, near real-time, or batch processing modes. In real-time operation, sensor data is processed continuously to provide immediate sleep stage classification and feedback.

In certain embodiments, batch processing may be used for retrospective analysis, enabling more computationally intensive processing. The system may dynamically switch between modes based on available resources or user preferences.

The embodiments described herein may be implemented using combinations of hardware, software, firmware, or microcode, and may be embodied in one or more non-transitory computer-readable media storing instructions that, when executed by one or more processors, processing circuits, or computing systems, cause the system to perform the operations described herein. The hardware components described herein may include discrete components, integrated circuits, application-specific integrated circuits (ASICs), field-programmable gate arrays (FPGAs), system-on-chip (SoC) architectures, or distributed computing components arranged across multiple devices. The processing operations described herein may be implemented using dedicated circuitry, programmable logic, or combinations of hardware and software executed by general-purpose or specialized processors.

It will be understood that the described systems may operate across heterogeneous computing environments including combinations of wearable devices, mobile devices, smartphones, augmented reality (AR) devices, virtual reality (VR) devices, mixed reality (MR) devices, IoT devices, edge computing devices, servers, and cloud computing platforms. Communication between such devices may occur using a variety of communication protocols, network topologies, and addressing schemes, including packet-based communication, streaming protocols, publish-subscribe architectures, or peer-to-peer communication frameworks. In certain embodiments, devices may be uniquely identified and addressed using identifiers such as IP addresses, MAC addresses, device identifiers, or cryptographic identifiers.

Input devices and sensors may include any devices capable of generating signals representative of physical, environmental, biological, user, or object states, and such signals may include electrical signals, optical signals, acoustic signals, electromagnetic signals, or digital representations thereof. The system may further include computer vision and object recognition components configured to process image, video, or sensor data to detect, classify, track, or identify objects, persons, gestures, or environmental features using feature extraction techniques, pattern recognition algorithms, or machine learning models.

The arrangement of components, data structures, processing steps, models, and system architectures described herein is provided for purposes of illustration and enabling disclosure, and alternative configurations, combinations, substitutions, and modifications may be implemented without departing from the scope of the invention. The described embodiments are intended to encompass implementations in which input data is acquired from one or more physical sources, transformed into structured data representations, processed using computational models, and used to generate outputs that correspond to a practical application, technical improvement, or control of a system or device. Accordingly, the scope of the invention is defined by the claims and is not limited to the specific embodiments described herein.

The foregoing has described the principles, embodiments, and modes of operation of the present invention. However, the invention should not be construed as being limited to the particular embodiments discussed. The above-described embodiments should be regarded as illustrative rather than restrictive, and it should be appreciated that variations may be made in those embodiments by workers skilled in the art without departing from the scope of the present invention as defined by the following claims.

What is claimed is:

1. A computer-implemented method for determining sleep states of a subject, comprising:
receiving, via one or more sensors of a wearable device, multimodal physiological data including at least photoplethysmography (PPG) signals and motion signals;
segmenting the multimodal physiological data into a plurality of primary time intervals;
determining, by one or more processors, a duration of each primary time interval using an artificial intelligence model trained on a historical sleep profile of the subject;
detecting, within a first primary time interval, a physiological transient event based on a correlation or divergence between a PPG-derived physiological metric and a motion-derived metric exceeding a dynamically determined threshold;
re-segmenting the first primary time interval into at least one sub-epoch window centered on the physiological transient event, wherein the sub-epoch window has a duration shorter than the primary time interval;
calculating a signal quality metric for at least the PPG signals within the sub-epoch window;
extracting features from the sub-epoch window and generating a plurality of sleep state probability distributions for the sub-epoch window;
modifying the plurality of sleep state probability distributions by scaling contributions of features derived from the PPG signals based on the signal quality metric; and
assigning a sleep state to the sub-epoch window based on the modified plurality of sleep state probability distributions.

2. The method of claim 1, wherein the artificial intelligence model dynamically adjusts the duration of the primary time intervals based on predicted sleep stages derived from the historical sleep profile.

3. The method of claim 1, wherein the duration of the sub-epoch window is decreased in response to an increase in signal entropy associated with the physiological data.

4. The method of claim 1, wherein the duration of the sub-epoch window is increased during a detected physiological steady-state condition.

5. The method of claim 1, wherein the signal quality metric is based on at least one of signal-to-noise ratio, motion artifact detection, sensor contact quality, or signal stability.

6. The method of claim 1, wherein modifying the plurality of sleep state probability distributions comprises attenuating feature contributions associated with signals having a signal quality metric below a threshold.

7. The method of claim 1, further comprising adjusting an operational parameter of at least one sensor of the wearable device based on the assigned sleep state.

8. A system for sleep stage classification, comprising:
a wearable sensor array configured to collect physiological data;
one or more processors; and
a memory storing instructions that, when executed by the one or more processors, cause the one or more processors to:
generate an initial sleep stage sequence based on the physiological data and a global transition probability model;
receive a timestamped user-generated event signal associated with a user-perceived or system-assisted sleep-related event;
define a temporal window surrounding a timestamp associated with the user-generated event signal;
generate a localized transition probability model specific to the temporal window, wherein the localized transition probability model modifies transition probabilities relative to the global transition probability model only within the temporal window; and
update the initial sleep stage sequence by executing a probabilistic sequence optimization process over the temporal window using the localized transition probability model to determine a most-likely sequence of hidden sleep states,
wherein the updating of the sleep stage sequence is performed without modifying the global transition probability model outside the temporal window, and
wherein the user-generated event signal is applied as a constraint on a probabilistic inference process.

9. The system of claim 8, wherein the probabilistic sequence optimization process comprises a probabilistic sequence optimization algorithm configured to determine a most-likely sequence of sleep states.

10. The system of claim 8, wherein the probabilistic sequence optimization algorithm comprises a Viterbi algorithm configured to maximize a joint probability of the sleep stage sequence given the localized transition probability model.

11. The system of claim 8, wherein the probabilistic sequence optimization algorithm comprises at least one of a forward-backward algorithm, a Baum-Welch algorithm, a conditional random field decoding process, or a neural sequence model.

12. The system of claim 8, wherein the temporal window spans at least 20 minutes surrounding the timestamp associated with the user-generated event signal.

13. The system of claim 8, wherein the localized transition probability model assigns increased transition likelihoods to sleep stage transitions corresponding to the user-generated event signal.

14. The system of claim 8, wherein the localized transition probability model weights the user-generated event signal based on a confidence level associated with the event.

15. A computer-implemented method for identifying non-standard physiological states, comprising:
mapping multimodal physiological data associated with a subject into a feature space;
identifying a latent sleep state cluster within the feature space, wherein the latent sleep state cluster represents a physiological condition not defined by predefined sleep stages;
determining that a feature vector corresponding to a current time interval is within a threshold distance of the latent sleep state cluster;
triggering a hardware state change in a wearable device in response to identifying the latent sleep state; and
recording the current time interval as corresponding to the latent sleep state.

16. The method of claim 15, wherein the threshold distance comprises a Mahalanobis distance.

17. The method of claim 15, wherein the hardware state change comprises at least one of increasing sensor sampling rate, increasing signal resolution, or activating a haptic actuator.

18. The method of claim 15, further comprising requiring the latent sleep state to persist for a minimum duration prior to triggering the hardware state change.

19. The method of claim 15, wherein the hardware state change is progressively adjusted based on a duration or recurrence of the latent sleep state.

20. The method of claim 15, wherein identifying the latent sleep state cluster comprises applying at least one of clustering, probabilistic modeling, or representation learning.
